(19) United States
(12) Patent Application Publication
(10) Pub. No.: US 2023/0082692 A1
(43) Pub. Date: Mar. 16, 2023

(54) Surgical Article And Method For Managing Surgical Articles During A Surgical Procedure

(71) Applicant: Stryker Corporation, Kalamazoo, MI (US)

(72) Inventors: Fazel Yavari, Portage, MI (US); Glen David Rocque, Portage, MI (US); Justin Andrews, Schoolcraft, MI (US); Brian James VanDerWoude, Portage, MI (US); Grant Westphal, Delton, MI (US); Nathan Hogan, Vicksburg, MI (US); Skylar Lee Clark, Augusta, MI (US)

(73) Assignee: Stryker Corporation, Kalamazoo, MI (US)

(21) Appl. No.: 17/992,486

(22) Filed: Nov. 22, 2022

Related U.S. Application Data
(63) Continuation of application No. 16/635,417, filed on Jan. 30, 2020.
(60) Provisional application No. 62/541,415, filed on Aug. 4, 2017.

(51) Int. Cl. A61B 90/98; A61B 50/37; A61B 90/00

(52) U.S. Cl. CPC A61B 90/98; A61B 50/37; A61B 90/08; A61B 2050/375; A61B 2090/0805

(57) ABSTRACT

Apparatus and method of locating and/or counting one or more surgical articles, wherein the surgical article may comprise a surgical sponge comprising a counting element and/or detecting element. The counting element and/or detecting element may comprise an RFID tag encapsulated in a housing or pouch that is coupled to the surgical sponge. The RFID tag may further comprise unique identification and/or location information relative to the surgical sponge. The RFID tag may be coupled to the sponge at an attachment position and/or oriented on the surgical sponge relative to a defined set of reference lines. The attachment position and/or orientation of the RFID tag on the surgical sponge may be configured to improve the accuracy of scanning the RFID tag. A plurality of surgical articles (i.e. surgical sponges) comprising RFID tags may be packaged or bundled via a strap or within a container.

### RELATED APPLICATIONS

This patent application is continuation of U.S. patent application Ser. No. 16/635,417, filed on Jan. 30, 2020, which is a National Stage Entry of International Patent Application No. PCT/US2018/045142, filed on Aug. 3, 2018, which claims priority to and the benefit of U.S. Provisional Application No. 62/541,415, filed on Aug. 4, 2017, the contents of which are hereby incorporated by reference in their entirety.

### TECHNICAL FIELD

The embodiments disclosed herein relate, generally, to surgical object detection and identification and, more specifically, to RFID tagged surgical sponges.

### BACKGROUND

Before and after a surgical procedure, it is important to track the tools and surgical articles utilized during the procedure to ensure proper sterilization and disposal of the tools and/or articles. It is also important to have an accurate count of the tools and/or articles to ensure that none of the tools and/or article were inadvertently lost or retained inside a patient. A surgical sponge is an example of a surgical article, which may be comprised of absorbent material for soaking up blood and other bodily fluids in and around an incision site. Health care professionals (HCPs) typically follow strict procedures to account for each and every sponge used during a surgery, in view of the risks associated with a surgical sponge being inadvertently retained inside a patient.

In the past, HCPs have relied upon counting surgical sponges by hand, however, manual counting requires handling of and exposure to soiled sponges and is prone to human error. To reduce the potential for retained surgical sponges associated with inaccurate manual counting methods, surgical sponges have been tagged with radio-opaque markers, barcodes, and/or wireless transponders, such as RFID or LC respondent transponders. However, when multiple surgical sponges are packaged in a single container, there can be interference with reading or acquiring an RFID signal from each individual sponge. Therefore, it is desirable provide a transponder on the surgical articles and/or method of packaging the surgical sponges to allow for efficient and accurate counting of the surgical sponges to reduce or eliminate the risks associated with surgical articles being retained inside a patient and the risks associated with the transmission of blood borne diseases.

### BRIEF DESCRIPTION OF THE DRAWINGS

Referring now to the drawings, exemplary illustrations are shown in detail. Although the drawings represent schematic embodiments, the drawings are not necessarily to scale and certain features may be exaggerated to better illustrate and explain an innovative aspect of an illustrative embodiment. Further, the exemplary illustrations described herein are not intended to be exhaustive or otherwise limiting or restricting to the precise form and configuration shown in the drawings and disclosed in the following detailed description.

Advantages of the present invention will be readily appreciated as the same becomes better understood by reference to the following detailed description when considered in connection with the accompanying drawings wherein:

FIG. 1 is a perspective view of a surgical sponge comprising an RFID tag.

FIG. 2A is a top view of the surgical sponge of FIG. 1 comprising an RFID tag.

FIG. 2B is a top view of the surgical sponge of FIG. 1 comprising an RFID tag.

FIG. 2C is a schematic view of the surgical sponge of FIG. 1 comprising a set of reference lines that intersect at an origin point.

FIG. 2D is a top view of the surgical sponge of FIG. 1 comprising a set of reference lines that intersect at an origin point and an RFID tag comprising a longitudinal axis.

FIG. 3A is a perspective view of a bundle of surgical sponges including a master tag.

FIG. 3B is a sectional view of the master tag of FIG. 3A.

FIG. 4 is a perspective view of a container for storing surgical sponges.

FIG. 5A is a perspective view of an example embodiment of a packaging configuration for a plurality of surgical sponges with an RFID tag.

FIG. 5B is a schematic of the example embodiment of the packaging configuration of FIG. 5A.

FIG. 6A is a perspective view of an alternative embodiment of a packaging configuration for a plurality of surgical sponges with an RFID tag.

FIG. 6B is a schematic of the alternative embodiment of the packaging configuration of FIG. 6A.

FIG. 7A is a perspective view of an alternative embodiment of a packaging configuration for a plurality of surgical sponges with an RFID tag.

FIG. 7B is a schematic of the alternative embodiment of the packaging configuration of FIG. 7A.

FIG. 8A is a perspective view of an alternative embodiment of a packaging configuration for a plurality of surgical sponges with an RFID tag.

FIG. 8B is a top view of the alternative embodiment of the packaging configuration of FIG. 8A.

FIG. 9A is a perspective view of an alternative embodiment of a packaging configuration for a plurality of surgical sponges with an RFID tag.

FIG. 9B is a top view of the alternative embodiment of the packaging configuration of FIG. 9A.

FIG. 10A is a perspective view of an alternative embodiment of a packaging configuration for a plurality of surgical sponges with an RFID tag.

FIG. 10B is a top view of the alternative embodiment of the packaging configuration of FIG. 10A.

FIG. 11 is graph illustrating the relationship between the distance between RFID tags and the effective read distance relative to the resonant frequency of the RFID tags.

FIG. 12 is a circuit diagram of an antenna tuning circuit.

FIG. 13 is an example table of a coding sequence for a rotary switch.

FIG. 14 is a printed circuit board assembly of the circuit shown in FIG. 12.

FIG. 15 is a perspective view showing the relative positioning of a tagged package to a scanning device.

FIG. 16 is a perspective view showing the relative positioning of a tagged object to a scanning device.

### DETAILED DESCRIPTION

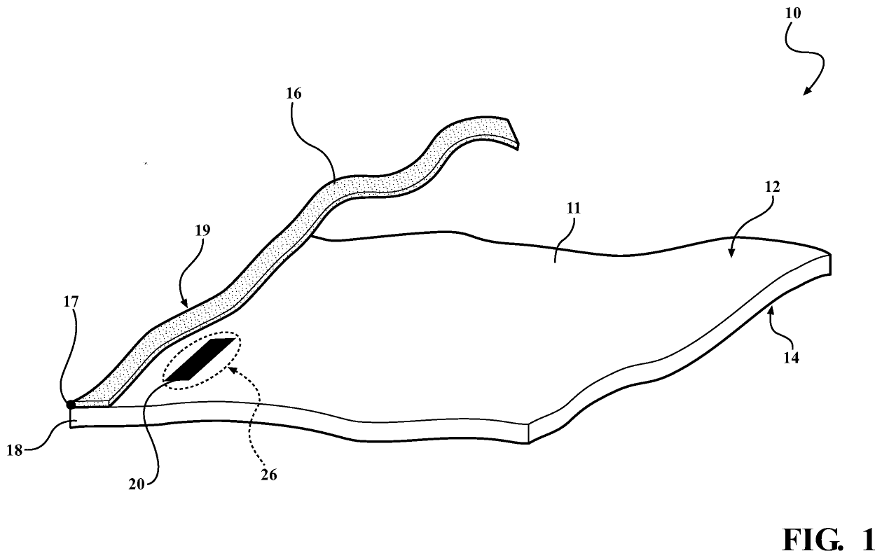

The present disclosure relates to a surgical article having one or more tags for counting and/or detecting the surgical article before, during, and/or after a surgical procedure. In particular, an embodiment of the surgical article illustrated in FIG. 1 may comprise a surgical sponge 10 further comprising a tag 20 as described in detail below. However, while not shown in the Figures, it has been contemplated that other embodiments of the surgical article 10 can include laparotomy pads, gauzes, implants, towels, suture needles, clips, staples, or surgical instruments. For example, another embodiment of the surgical article may comprise a surgical instrument, such as a scalpel or forceps, comprising a tag 20. The tag 20 may comprise counting element(s), detecting element(s), or any combination thereof and may be incorporated within handles, between layers of, and/or other portions of the surgical article 10. As described in detail below, each surgical article 10 can include one or more tags 20, and each tag 20 may include various combinations of the counting elements and/or the detecting elements. For example, one of these tags 20 may comprise a RFID element (RFID tag) and/or an optically-scannable element configured to allow for detection of and/or counting the surgical article(s). However, each tag 20 can include any number of counting elements and any number of detecting elements.

A tag 20 comprising a counting element and/or detecting element may be configured to include unique identification information for each surgical article 10. The unique identification information may comprise a serial number or other identifier that is unique and assigned only to the corresponding article 10. In other embodiments, the unique identification information may further convey the type, size, weight, manufacturing dates, expiration date, number of similar articles 10 in a corresponding pack, and/or other information used for counting or detecting the article 10.

The tag 20 may convey unique identification information by transmitting an electromagnetic signal or wave corresponding with the unique identification information. Each surgical article 10 may comprise a tag 20 with a unique identifier that is scannable by an optical-scanning device or can be manually entered into a user interface sub-assembly of the scanning device, computer, or other system. The plurality of tags 20 on each surgical article 10 may be different from one another, yet include the same unique identification information related to the specific surgical article 10. The tag(s) 20 may allow an HCP to identify the number of surgical articles 10 present and/or to determine a location of the surgical articles 10 within the body of the patient, within an operating room, or both inside the body of the patient and within the operating room. In certain other embodiments, the tag(s) 20 may be detectable within the operating room but not within the body of the patient.

The scanning device may be configured to maintain a record of the surgical articles 10 used in the procedure in on-board memory, or in cooperation with a server. The scanning device includes an RFID interrogator in communication with the on-board memory. The RFID interrogator includes the physical components and the operating software for generating and receiving the radio frequency signals. Among the physical components of the interrogator are the radio controller, including a signal-generating transmitter, a signal receiver, or a transceiver. The interrogator further includes an antenna. An antenna tuning circuit may also be provided between the antenna and the radio controller. The scanning device, through the radio controller in communication with the tuning circuit and the antenna, communicates with the RFID tags to retrieve the information stored on the tags and generate a record of the articles 10 used in the procedure. The record may be stored on the scanning device in on-board memory, or else the record may be communicated to a server for storage. The scanning device may have a wired or a wireless connection to the server. In some alternatives, there may be one or more devices disposed in communication between the scanning device and the server. In one example, the scanning device within an operating room may communicate with a computer located in the operating room that is in further communication with other medical devices and tools in the operating room. The computer may communicate information to a router that acts as a gateway to a network. The server is also connected to the network, and thus the scanning device communicates with the server through multiple layers of devices.

At the conclusion of the surgical procedure, the records can be transmitted to a server and matched with patient records, such as electronic medical records, to update the same and provide an indication of which specific surgical articles were used with each patient at which times.

The tag 20 may be incorporated within handles, between layers of, and/or other portions of the surgical article 10. For example, the tag 20 can be adhered to or encapsulated within the layers of the surgical article 10, embedded within the handle, or coupled to other portions of the article 10. In one embodiment, the surgical article 10 can have diametrically opposite sides or corners, and two tags 20 comprising detecting elements may be coupled to those opposing corners of the article 10 so as to space apart the tags 20 by a maximum distance, and thus reduce the probability of both tags being damaged and rendered undetectable. However, it is contemplated that the tags 20 may be disposed adjacent to one another or coupled to any suitable portion of the article. Each tag 20 may be rigid to increase its service life. In other embodiments, the tag 20 can be flexible to permit the surgical article 10 and the tag to be folded or otherwise shaped in a more preferable manner for use within a patient's body. Furthermore, the tag 20 may be encapsulated in a biocompatible plastic coating, pouch, or housing 26 that is water-impermeable and sterilizable. The housing 26 may be coupled to the surgical article 10 via stitching, adhesive, or similar type of fastener.

The counting and/or detecting elements of the tag 20 may be configured to cooperate with at least one remote detector-interrogating antenna (detecting antenna) of a reader, as a scanning device, such as a hand-held wand manipulated by the HCP. Alternatively, remote detector-interrogating antenna may be incorporated into a surgical instrument tray, surgical cart, and/or canister. However, it is contemplated that any suitable antenna, including one integrated within the optical-scanning device can be configured to detect the detecting element included in the tag 20. The antenna may further comprise a circuit, coil, or loop configured to define a plane of the antenna, wherein a signal, which can be carried on, or understood as, an electromagnetic field, may be transmitted outward from the plane of the antenna, to be received by the tags 20 which then provides a response signal that can be projected back to the antenna. The Applicant has described a scanning device or scanning apparatus with an antenna in U.S. Pat. No. 8,181,860, filed on Sep. 13, 2007, the disclosure of which is hereby incorporated by reference.

A wide variety of tags may be commercially available by a number of manufacturers. Certain tags may be configured to provide significant amounts of user accessible memory, sometimes in the form of read-only memory or write-once memory. One exemplary tag is an RFID tag 20 detectable by a RFID antenna. However, it is contemplated that the surgical article 10 can include any suitable tag detectable by any corresponding detecting antenna. The Applicant has described a surgical article 10 and method of managing surgical articles that comprise various tags in PCT Application No. PCT/US2016/057077, filed on Oct. 14, 2016, the disclosure of which is hereby incorporated by reference.

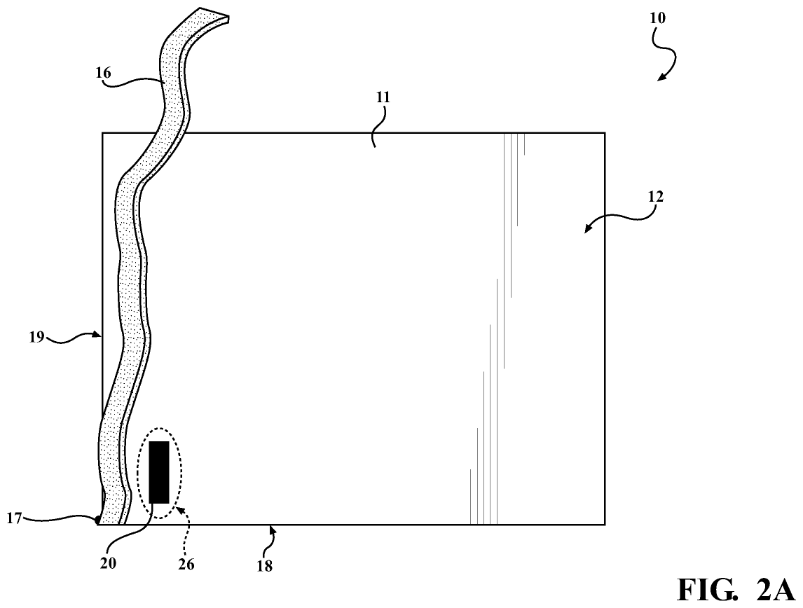

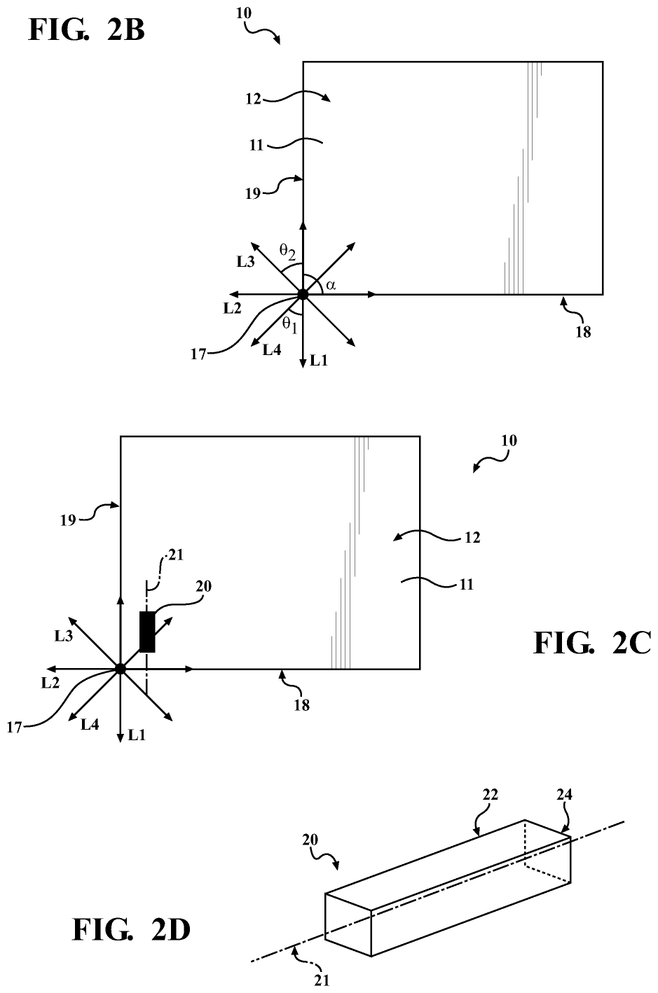

Referring to FIGS. 1 and 2A-2C, an example embodiment of a surgical article 10 is illustrated. The surgical article may comprise a surgical sponge 10 comprising an absorbent material body 11. The absorbent material body 11 of the surgical sponge 10 may comprise a top surface 12 and an opposing bottom surface 14. The surgical sponge 10 may further comprise a lead, handle or string 16. The lead 16 may comprise a radio opaque marker material that is configured to show up in a medical scan. For example, the lead may comprises a radio opaque marker material configured to show up in a MRI image to allow for identification of a surgical sponge 10 that was inadvertently retained within a patient.

It may be critical for HCPs to track surgical sponges 10 before, during, and after a surgical procedure to ensure that a surgical sponge 10 is not inadvertently retained or left within a patient. Therefore, as described above, an RFID tag 20 may be utilized to identify the location and number of sponges used in a surgical procedure. An RFID tag 20 may be coupled to the top surface of the absorbent material body 11 proximate 12 to an edge or corner of the surgical sponge 10. While not shown in the Figures, it is contemplated that the RFID tag 20 may be incorporated into the handle, between layers of the absorbent material or other portions of the surgical sponge 10 in any number of ways. For example, the RFID tag 20 can be adhered to or embedded within the handle, or coupled to other portions of the surgical sponge 10. The RFID tag 20 may be encapsulated in a biocompatible plastic coating, pouch, or housing 26 that is water-impermeable and sterilizable. The housing 26 may be coupled to the surgical article 10 via stitching, adhesive, or similar type of fastener. It is also contemplated that a plurality of RFID tags 20 may be coupled to the same surgical sponge 10. For example, in one embodiment, the surgical sponge 10 can have diametrically opposite sides or corners, and two RFID tags 20 may be coupled to those opposing corners of the article. Alternatively, RFID tags 20 may be coupled to opposing surfaces 12, 14 of the surgical sponge 10. For example, a first RFID tag 20 may be attached to the top surface 12 of the surgical sponge 10, and a subsequent RFID tag 20 may be coupled to the bottom surface 14 of the surgical sponge 10.

The RFID tag 20 may be configured in a variety of shapes and sizes. As illustrated in FIG. 2D, the RFID tag 20 is configured in the shape of a rectangle, having an elongated side 22 and narrow side 24, relative to one another. The RFID coil or antenna (not shown) may be oriented to extend along the longitudinal or elongate side 22 of the RFID tag 20, and may define a longitudinal axis 21 of the RFID tag 20. The longitudinal axis 21 is illustrated along the long dimension of the rectangular tag 20, however, this is not limiting. While not shown in the Figures, it is further contemplated that the RFID tag 20 may configured in any size and/or shape. For example, the RFID tag 20 may be configured in a square or other polygonal, circular, or oval shape. As used herein reference to a longitudinal axis refers to an orientation of field lines of the electromagnetic communication between the scanning device and the tags and is not limited to any particular shape, position or orientation of the RFID tag or antenna.

The RFID tag 20 coupled to the surgical sponge 10 or similar surgical article may be encapsulated in a biocompatible plastic coating, pouch, or housing 26 that is water-impermeable and sterilizable. Each RFID tag 20 may be rigid to increase its service life. In other embodiments, the tag containing the RFID tag 20 can be flexible to permit the surgical article and the tag to be folded or otherwise shaped in a more preferable manner for use within a patient's body. RFID tag 20 and housing 26 may be attached or coupled to the surgical sponge via stitching, glue, sewing, woven fabric, or other similar fasteners. For example, the housing 26 containing the RFID tag 20 may be sewn to the top surface 12 of the absorbent material body 11 of the surgical sponge 10. Alternatively, the housing 26 may be attached to the absorbent material body 11 of the surgical sponge by an adhesive, such as a glue or epoxy. In the preferred embodiment the RFID tag 20 is encapsulated in a biocompatible plastic housing that is water-impermeable and sterilizable, which is enclosed in a woven fabric via stitching, which is then attached to the absorbent material body 11 of the surgical sponge 10 by stitching.

The detecting element of the RFID tag 20 may be used with a multiplex detection system. Continuing with the previous embodiment, the RFID tag 20 can include a capacitor and an antenna (not shown), which receives power from the detecting antenna (RFID antenna) of the reader to charge the capacitor of the RFID tag 20. This capacitor becomes the power source for the operation of the unpowered RFID tag 20. The RFID tag 20 can have an integrated circuit, which includes a reading function, a carrier frequency modulating function, and a read-only memory portion with a burned-in code. The integrated circuit and corresponding antenna of the detecting element are encapsulated in an enclosure that is resistant to blood, water, or saline solution. Thus, the RFID tag 20 can withstand repeated sterilization and be attached to other surgical articles, such as metal instruments, which are sterilized and reused multiple times. Depending on the carrier frequency and the type of RFID tag 20, the RFID tag 20 can vary significantly in cost, size, and resistance to shielding by intervening tissue.

While other detecting or counting elements may only permit detection of the location, one of the advantages provided by RFID based technology is that the RFID tag 20 achieves the dual purpose of detecting the location of the surgical sponge 10 in addition to counting and/or identifying the surgical sponge 10. Thus, certain RFID tags 20 may serve as both detection elements and counting elements. The RFID tag cooperates with the detecting antenna of the reader to both detect the location of the surgical sponge 10 and provide data for determining the unique identification information of the surgical sponge 10. Some embodiments of the RFID tags 20 may operate above the MHz range. Exemplary frequencies can include about 13.35 to 14.15 MHz (high frequency), a range from 850 to 950 MHz (ultra-high frequency), or a range of microwave frequencies (i.e., 2.45 to 2.55 GHz). The added bandwidth provided by these RFID tags 20 can increase the probability of detecting and finding the corresponding surgical sponge 10 within the interrogation zone and within a short period of time. For example, the RFID tag 20 of adjacent sponges 10 may be configured to operate on different frequencies to prevent interference and further improve the accuracy of counting and/or identifying the surgical sponges 10. This will allow a number of surgical sponge 10 to be identified and inventoried near simultaneously, such as the surgical sponge 10 lying on the instrument table, or multiple surgical sponge 10 contained within a bodily cavity of the patient.

To accomplish the dual purpose of being detectable and transmitting the unique identification information, some embodiments of the RFID tag 20 can include: the LF Ferrite rod; HF Ferrite rod; HF label element; UHF Ferrite element; UHF label element; and/or combinations thereof. Thus, in certain configurations, one or more of the detecting elements included in the article may be detectable by multiple radio detection modalities. The RFID tag 20 can be a bead of ferrite with a coil configured to resonate at a designated frequency. Alternatively, the RFID tag 20 can be a flexible thread composed of a single loop wire and capacitor element. The detecting antenna of the reader can locate the RFID tag 20 by pulsed emission of a wide-band transmission signal, and the RFID tag 20 can resonate with a radiated signal, in response to the wide band transmission, at its own single non-predetermined frequency within the wide band range.

In certain embodiments, the RFID tag 20 may utilize a frequency range of from 10 MHz to 1 GHz. Examples of the RFID tag include an LF Ferrite rod, HF Ferrite rod, HF label element, UHF Ferrite element, and/or UHF label element. In these embodiments, the RFID tag 20 may transmit a signal or wave to a system to provide the unique identification information of the article. The RFID tag 20 may convey the same unique identification information provided by the optically-scannable element and the human-readable element described above.

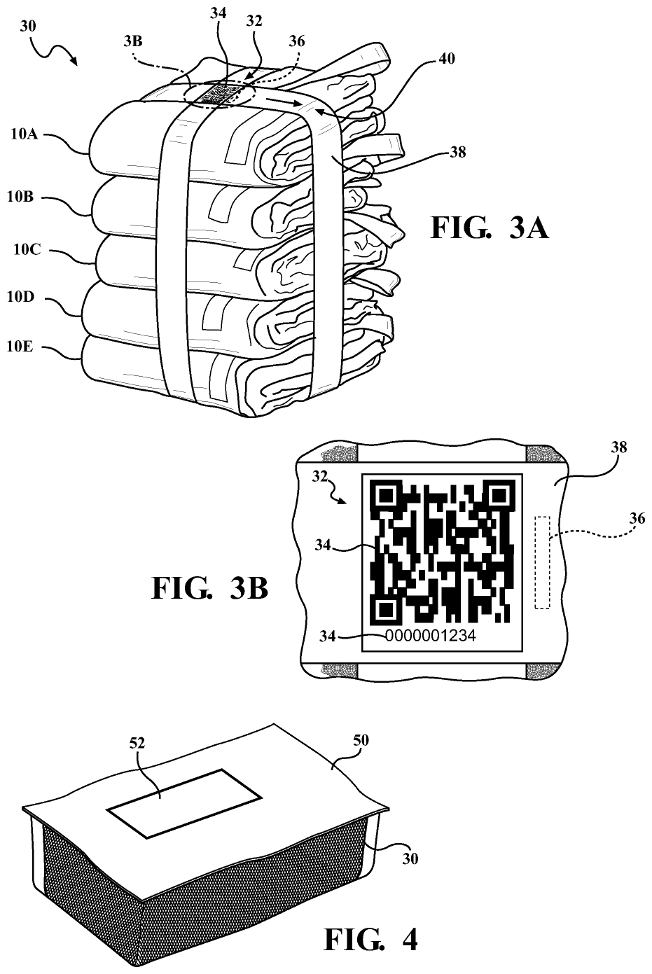

FIGS. 3A, 3B, and 4 illustrate example embodiments of various containers for packaging or bundling two or more surgical sponges 10. In one embodiment, as illustrated in FIG. 3A, a strap 38 or plurality of straps 38 may be utilized to bundle or package two or more surgical sponges 10 together. The strap(s) 38 may be configured to bundle the two or more surgical sponges 10 to maintain a defined relationship between the RFID tags 20 of adjacent surgical sponges 10 in the bundle 30. Alternatively, two or more surgical sponges 10 may be packaged or bundled within a pouch or container 50. The container 50 may be similarly configured to the strap, wherein the container 50 is configured to maintain a defined relationship between the RFID tags 20 of adjacent surgical sponges 10 in the container 50. The container 50 may comprise a poly-Tyvek pouch, a rigid base with a poly-Tyvek cover, or similar containment apparatus. Any number of surgical sponges 10, or surgical instruments may be packaged or bundled by the strap 38 or within the container 50. For example, a plurality of surgical sponges 10 may stacked on top of one another and packaged together by a strap or band 38. The strap 38 may be configured to bundle 2, 3, 5, 10, 20, or more surgical sponges 10 together.

The bundle 30 may comprise a master tag 32 that may include unique identification information 34, 38. For example, the master tag 32 may include the number of surgical sponges 10 included in the bundle 30, as well as include the unique identification information for each of the sponges 10 contained in the bundle 30.

The master tag 32 may be configured to identify the side of bundle 30 and/or package/object that should be scanned based on the position of the RFID tags 20. For example, the master tag 32 or the packaging 38 may include an indicator 40 to facilitate such placement/orientation, such as an arrow pointing to the side of the bundle 30 that should be scanned for optimal accuracy. Alternatively, an indicator 40 could be placed on the side of the bundle that should be placed closest to the scanner. The indicator 40 may be placed on one or multiple sides of the bundle of sponges 30. The indicator 40 may be placed on any side/face of the bundle.

The indicator 40 may also indicate the optimal direction of moving the bundle and/or object in relationship to the scanner. For example, the indicator 40 could signal to the HCP that they should move the bundle 30 parallel to the plane of the scanner, around the perimeter of the scanner, rotate the bundle 30 in front of the scanner or any other method of movement that could optimize the scanning of the bundle 30. A container 50 configured to hold an individual or plurality of bundles of surgical sponges 30 may comprise a similar master tag 52 configured to identify the contents of the container. For example, the container master tag 52 may identify the contents of the container 50, such as the number of bundles 30 included in the container 50, as well as any additional equipment or medical instruments included in the container 50. A container 50 configured to hold a bundle of surgical sponges 30 may comprise a similar indicator configured to identify optimal scanning orientation/position/movement.

Bundling or packaging surgical sponges 10 as described above can create challenges for scanning the RFID tag(s) 20 of the individual surgical sponges 10 included in the bundle 30. When attempting to scan the RFID tag 20 of the surgical sponges 10 when packaged, one potential challenge is that there is an increased possibility of interference between the RFID tags 20 that may result in an inaccurate count or reduced detection distance of the surgical sponges 10. Providing a sufficient detection distance is important for surgical tools and articles because the sterility of the surgical tools and articles needs to be maintained in order to help prevent a patient infection. Preferably a HCP can scan the RFID tag while within the sterile field without a concern of contaminating the surgical tools and articles. If the read distance is insufficient the HCP will need to spend additional time and incur additional costs to sterilely drape the scanner prior to each surgery, or sterilize and maintain the sterility of the scanner between surgeries

A coordinate system including a plurality of lines configured to intersect at an origin point 17 may be utilized as a reference to describe the orientation of the RFID tag 20 relative to the surgical sponge 10. The coordinate system shown in FIGS. 2B and 2C, comprises 4 lines, a first line L1, a second line L2, a third line L3, and a fourth line L4. The origin point 17 where the lines L1, L2, L3, and L4 intersect may be placed proximate to a corner of the absorbent material body 11 of the surgical sponge 10, wherein the first line L1 extends along a first edge 19 of the absorbent material body 11, and the second line L2 extends along a second edge 18 of the absorbent material body 11. The first line L1 and the second line L2 may be configured to be intersect at an angle alpha α, as shown in FIG. 2B. Angle alpha α may be any angle ranging from zero degrees to 360 degrees when measured at the point where the first line L1 and second line L2 intersect. In a preferred embodiment, the first line L1 and second line L2 may be configured to be generally perpendicular to one another, wherein angle alpha α is approximately 75 degrees to 105 degrees. The first 18 and second 19 edges of the absorbent material body 11 may be created by stitching, folding, and/or terminal ends of the absorbent material body 11. The third line L3 and the fourth line L4 are configured to intersect with one another and/or to intersect with the first line L1. The origin 17 may be positioned at the point where the third line L3 and/or fourth line L4 intersect the first line L1. The third line L3 and/or fourth line L4 may be configured to intersect the first line L1 at an angle theta θ. Angle theta θ may be any angle ranging from zero degrees to 360 degrees when measured at the point of where the third line L3 and/or fourth line L4 intersect the first line L1. In a preferred embodiment, third line L3 and/or fourth line L4 may be configured to be generally perpendicular to one another, and wherein angle theta θ is approximately 30 degrees to 60 degrees as measured relative to the first line L1 (as shown in FIG. 2B).

There are a number of ways to improve the accuracy of scanning RFID tags 20 attached to surgical sponges 10, or other surgical articles that may be packaged together. For example, the rotational orientation of the RFID tag 20 relative to a defined point of origin 17 of the surgical sponge 10, wherein the RFID tags 20 of one or more adjacent surgical sponges 10 may be oriented differently. The attachment position of the RFID tags 20 may also be different for the one or more adjacent surgical sponges 10. Furthermore, the arrangement of the surgical sponge 10 within the packaging 38, 50 may be different for one or more adjacent surgical sponges 10. For example, an individual surgical sponge 10 may be flipped relative to one or more adjacent surgical sponges 10 within the packaging 38, 50. The arrangement of one or more adjacent surgical sponges 10 may similarly be rotated relative to the packaging 38, 50. The resonant frequency of individual RFID tags 20 may also be selected or assigned wherein the resonant frequency of the RFID tags 20 of one or more adjacent surgical sponges 20 will be different. It is contemplated that any individual embodiment may comprise a single configuration and/or combination of configurations described to improve the accuracy of scanning RFID tags 20. Various configurations and or combinations of surgical sponges 10 and RFID tags 20 will be explained in greater detail below. However, the following descriptions of the various configurations are not intended to be exhaustive or limiting, but rather intended to serve as example embodiments.

As described above, surgical sponges 10 may be stacked on top of one another as part of the packaging process. When stacked, the RFID tag 20 of one or more adjacent surgical sponges 10 will be on different planes. Therefore, the longitudinal axes 21 of the various RFID tags 20 of one or more adjacent surgical sponges 10 may be projected onto a common plane and coordinate system for the purpose of comparing and/or orienting the RFID tags 20 of adjacent surgical sponges 10.

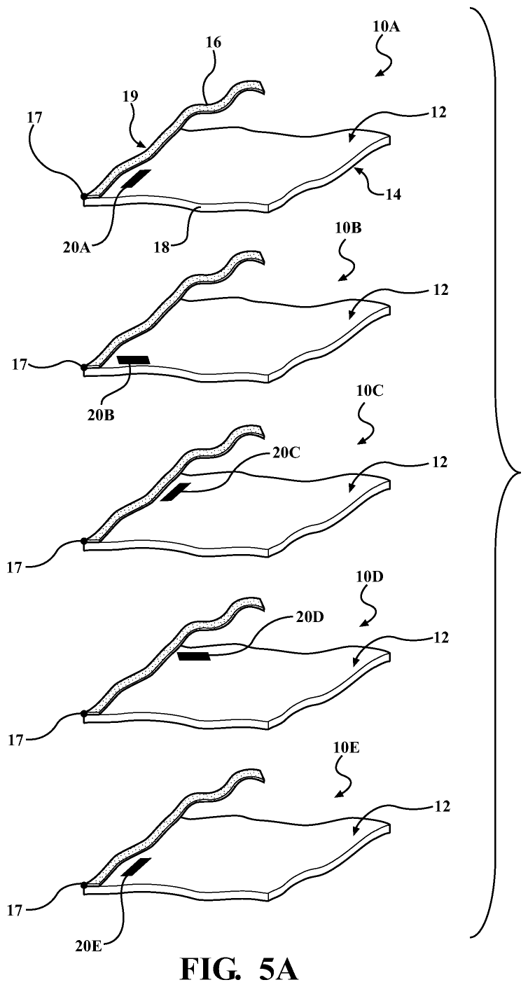

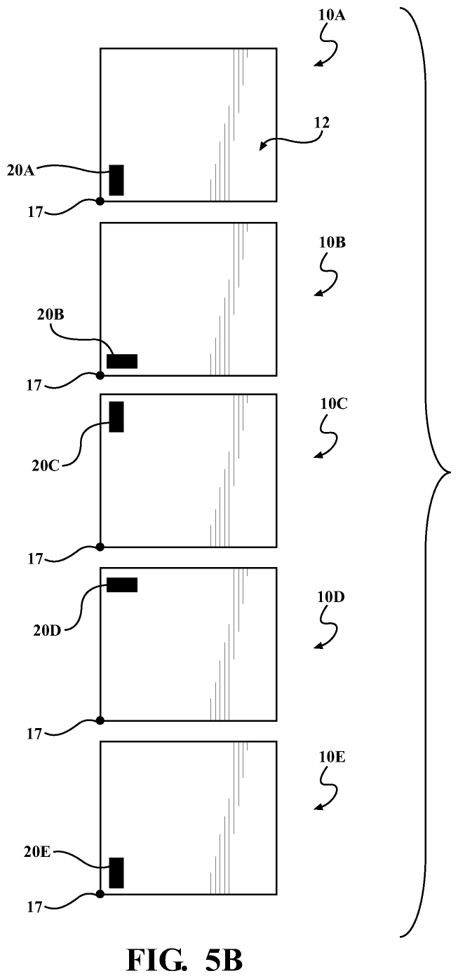

Referring to FIGS. 5A and 5B, an example embodiment of a configuration for packaging a plurality of surgical sponges 10 is illustrated. The RFID tag 20 may be attached to the absorbent material body 11 at an attachment position proximate the edge and/or corner of the absorbent material body 11 of the surgical sponge 10. To prevent interference between the RFID tags 20 of adjacent surgical sponges 10 that have been stacked during packaging, the orientation of the RFID tag 20 may be different. For example, as illustrated in FIGS. 5A and 5B, a first surgical sponge 10A may comprise a first RFID tag 20A attached at a first attachment position of the surgical sponge 10A with the longitudinal axis 21 of the RFID tag 20A oriented to be generally parallel to the first line L1. A second surgical sponge 10B, placed adjacent the first surgical sponge 10A when packaged, may comprise a second RFID tag 20B attached at a second attachment position of the surgical sponge 10B with the longitudinal axis 21 of the RFID tag 20B oriented to be generally parallel to the second line L2. When the first surgical sponge 10A and second surgical sponge 10B are stacked on top of one another and the various longitudinal axes 21 of each RFID tag 20A, 20B are projected onto a common plane, the longitudinal axis 21 of the first RFID tag 20A and the second RFID tag 20B will be oriented in generally perpendicular configuration relative to one another when viewed from above. One of a plurality of advantages provided by this configuration is the reduced likelihood of interference when scanning the first and second surgical sponges 10A, 10B while stacked, increasing the RFID tag read distance and decreasing the likelihood of an error in counting and/or locating surgical sponges before, during, and after a surgical procedure.

Additionally, the RFID tag 20 may be attached to adjacent or opposing corners of the surgical sponges 10 relative to the RFID tag 20 of an adjacent surgical sponge 10. For example, as illustrated in FIGS. 5A and 5B, a second surgical sponge 10B may comprise a second RFID tag 20B attached at the second attachment position of the surgical sponge 10B with the longitudinal axis 21 of the RFID tag 20B oriented to be generally parallel to the second line L2. A third surgical sponge 10C, placed adjacent the second surgical sponge 10B when packaged, may comprise a third RFID tag 20C attached at a third attachment position of the surgical sponge 10C with the longitudinal axis 21 of the RFID tag 20C oriented to be generally parallel to the first line L1. When the second surgical sponge 10B and third surgical sponge 10C are stacked on top of one another and the various longitudinal axes of each RFID tag 20 are projected onto a common plane, the longitudinal axis 21 of the second RFID tag 20B and the third RFID tag 20C will be oriented in generally perpendicular configuration relative to one another when viewed from above. Furthermore, the third RFID tag 20C of the third surgical sponge 10C will be in an adjacent corner (third attachment position) relative to the second RFID tag 20B of the second surgical sponge 10B (second attachment position).

When adding a fourth surgical sponge 10D to the stack, the orientation of the fourth RFID tag 20D may be different relative to the third RFID tag 20C of the third surgical sponge 10C. For example, as illustrated in FIGS. 5A and 5B, a third surgical sponge 10C may comprise a third RFID tag 20C attached at the third attachment position of the surgical sponge 10C with the longitudinal axis 21 of the RFID tag 20C oriented to be generally parallel to the first line L1. The fourth surgical sponge 10D, placed adjacent the third surgical sponge 10C when packaged, may comprise a fourth RFID tag 20D attached at a fourth attachment position of the surgical sponge 10 with the longitudinal axis 21 of the RFID tag 20A oriented to be generally parallel to the second line L2. When the third surgical sponge 10C and fourth surgical sponge 10D are stacked on top of one another and the various longitudinal axes 21 of each RFID tag 20C, 20D are projected onto a common plane, the longitudinal axis 21 of the third RFID tag 20C and the fourth RFID tag 20D will be oriented in generally perpendicular configuration relative to one another when viewed from above.

The orientation of the RFID tag 20 and/or the location of the RFID tag 20 on the surgical sponge 10 of adjacent surgical sponges 10 may be manipulated as described above to reduce interference when scanning for RFID tags 20 to count and/or locate the corresponding surgical sponges 10. When adding additional surgical sponges 10 to the stack, the orientation and/or attachment position of the RFID tag 20 of a prior surgical sponge 10 may be repeated in a subsequent surgical sponge 10, however, the orientation and/or attachment position of the RFID tag 20 on the adjacent surgical sponges 10 should be different. For example, referring to FIGS. 5A and 5B, a fifth surgical sponge 10E may comprise a fifth RFID tag 20E attached at a fifth attachment position of the surgical sponge 10E with the longitudinal axis 21 of the RFID tag 20E oriented to be generally parallel to the first line L1, similar to the first RFID tag 10A of the first surgical sponge 10A. However, the first surgical sponge 10A and the fifth surgical sponge 10E are not adjacent to one another when stacked and assembled in the packaging. The gap between the first and fifth surgical sponges 10A, 10E created by the intermediary surgical sponges 10B, 10C, 10D reduces the likelihood of interference between the first RFID tag 10A and fifth RFID tag 10E when scanned.

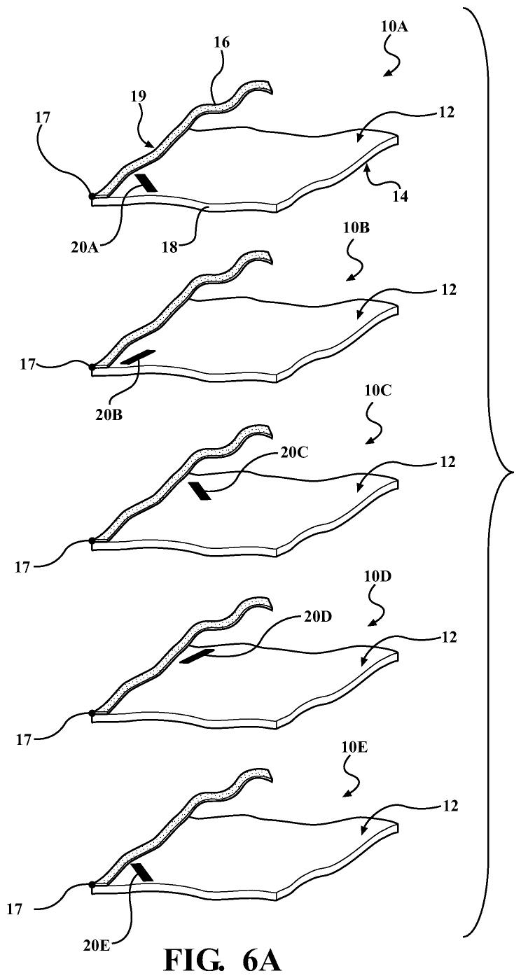

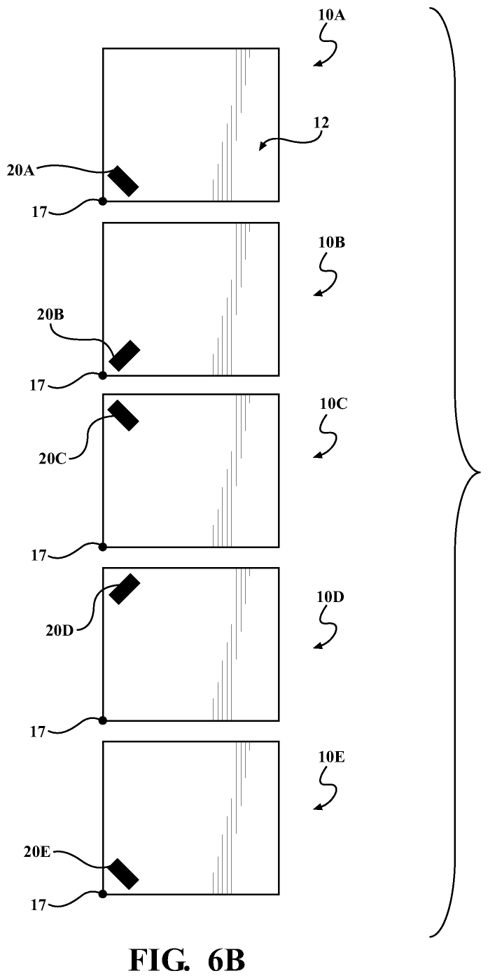

Referring to FIGS. 6A and 6B, an alternative embodiment of a configuration for packaging a plurality of surgical sponges 10 is illustrated. To prevent interference between the RFID tags 20 of adjacent surgical sponges 10 that have been stacked during packaging, the orientation of the RFID tag 20 may be different. For example, as illustrated in FIGS. 6A and 6B, a first surgical sponge 10A may comprise a first RFID tag 20A attached at a first attachment position of the surgical sponge 10A with the longitudinal axis 21 of the RFID tag 20A oriented to be generally parallel to the third line L3. A second surgical sponge 10B, placed adjacent the first surgical sponge 10A when packaged, may comprise a second RFID tag 20B attached at a second attachment position of the surgical sponge 10B with the longitudinal axis 21 of the RFID tag 20A oriented to be generally parallel to the fourth line L4. When the first surgical sponge 10A and second surgical sponge 10B are stacked on top of one another and the various longitudinal axes 21 of each RFID tags 20A, 20B are projected onto a common plane, the longitudinal axis 21 of the first RFID tag 20A and the second RFID tag 20B will be oriented in generally perpendicular configuration relative to one another when viewed from above.

Additionally, the RFID tag 20 may be attached to adjacent or opposing corners of the surgical sponges 10 relative to the RFID tag 20 of adjacent surgical sponges 10. For example, as illustrated in FIGS. 6A and 6B, a second surgical sponge 10B may comprise a second RFID tag 20B attached at the second attachment position of the surgical sponge 10B with the longitudinal axis 21 of the RFID tag 20B oriented to be generally parallel to the fourth line L4. A third surgical sponge 10C, placed adjacent the second surgical sponge 10B when packaged, may comprise a third RFID tag 20C attached at the third attachment position of the surgical sponge 10C with the longitudinal axis 21 of the RFID tag 20C oriented to be generally parallel to the third line L3. When the second surgical sponge 10B and third surgical sponge 10C are stacked on top of one another and the various longitudinal axes 21 of each RFID tags 20B, 20C are projected onto a common plane, the longitudinal axis 21 of the second RFID tag 20B and the third RFID tag 20C will be oriented in generally perpendicular configuration relative to one another when viewed from above. Furthermore, the third RFID tag 20C of the third surgical sponge 10C will be in an adjacent corner (third attachment position) relative to the second RFID tag 20B of the second surgical sponge 10B (second attachment position).

When adding a fourth surgical sponge 10D to the stack, the orientation of the fourth RFID tag 20D may be different relative to the third RFID tag 20C of the third surgical sponge 10C. For example, as illustrated in FIGS. 6A and 6B, a third surgical sponge 10C may comprise a third RFID tag 20C attached at the third attachment position of the surgical sponge 10C with the longitudinal axis 21 of the RFID tag 20C oriented to be generally parallel to the third line L3. The fourth surgical sponge 10D, placed adjacent the third surgical sponge 10C when packaged, may comprise a fourth RFID tag 20D attached at the fourth attachment position of the surgical sponge 10D with the longitudinal axis 21 of the RFID tag 20C oriented to be generally parallel to the fourth line L4. When the third surgical sponge 10C and fourth surgical sponge 10D are stacked on top of one another and the various longitudinal axes of each RFID tag 20 are projected onto a common plane, the longitudinal axis 21 of the third RFID tag 20C and the fourth RFID tag 20D will be oriented in generally perpendicular configuration relative to one another when viewed from above.

The orientation of the RFID tag 20 and/or the attachment position of the RFID tag 20 on the surgical sponge 10 of adjacent surgical sponges 10 may be manipulated as described above to reduce interference when scanning for RFID tags 20 to count and or locate the corresponding surgical sponges 10. For example, referring to FIGS. 6A and 6B, a fifth surgical sponge 10E may comprise a fifth RFID tag 20E attached at the fifth attachment position of the surgical sponge 10 with the longitudinal axis 21 of the RFID tag 20E oriented to be generally parallel to the third line L3, similar to the first RFID tag 10A of the first surgical sponge 10A. However, the first surgical sponge 10A and the fifth surgical sponge 10E are not adjacent to one another when stacked and assembled in the packaging. The gap between the first and fifth surgical sponges 10A, 10E created by the intermediary surgical sponges 10B, 10C, 10D reduces the likelihood of interference between the first RFID tag 10A and fifth RFID tag 10E when scanned.

The orientation of surgical sponges 30 as configured in FIGS. 6A and 6B. limits the number of RFID Tags 20 with coplanar longitudinal axes to a single pair, and the pair with coplanar longitudinal axes are separated by the intermediary surgical sponges 10B, 10C, and 10D. Coplanar longitudinal axes in RFID tags 20 in close proximity can have significant positive or negative effects on the read distance of the RFID tags 20 with coplanar longitudinal axes depending on the resonant frequency of the RFID tags 20. By intentionally separating the RFID tags with coplanar axes the effect of RFID tags with longitudinal coplanar axes on read distance is reduced allowing the surgical sponge manufacturing process to attach RFID tags 20 without presorting the RFID tags 20 based on the resonant frequency of each RFID tag 20.

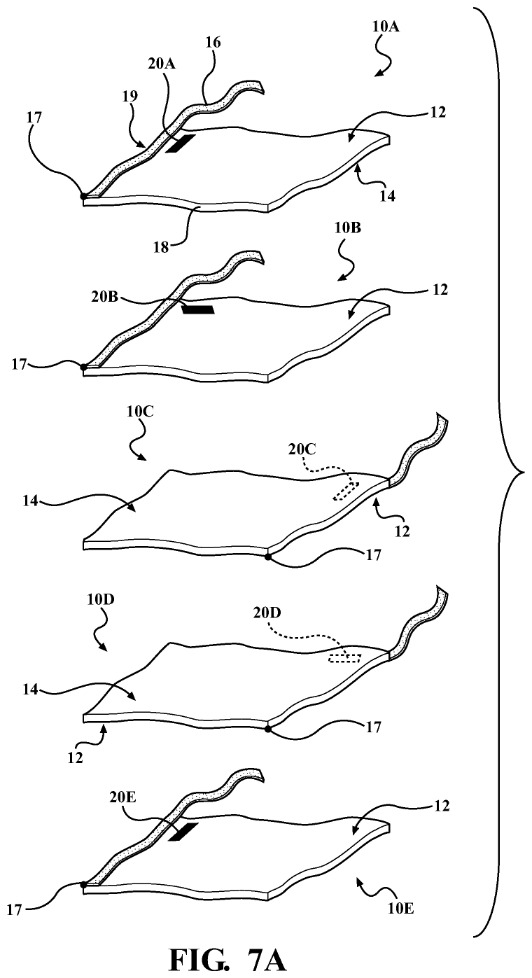

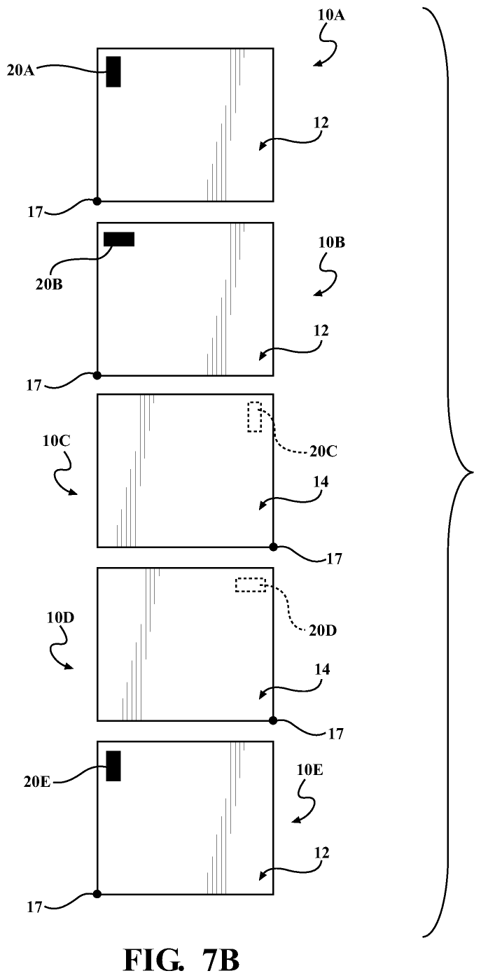

Referring to FIGS. 7A and 7B, an alternative embodiment of a configuration for packaging a plurality of surgical sponges 10 is illustrated. The RFID tag 20 may be attached to the absorbent material body 11 at an attachment position proximate the edge and/or corner of the surgical sponge 10. As discussed above, the orientation of the RFID tags 20 on adjacent surgical sponges 10 may be different. Additionally, the arrangement of the surgical sponge 10 may also be manipulated relative to the packaging to differentiate the attachment position and/or orientation of the RFID tag 20 of adjacent surgical sponges 10. For example, as illustrated in FIGS. 7A and 7B, a first surgical sponge 10A may comprise a first RFID tag 20A attached at the first attachment position of the surgical sponge 10A with the longitudinal axis 21 of the RFID tag 20A oriented to be generally parallel to the first line L1. A second surgical sponge 10B, placed adjacent the first surgical sponge 10A when packaged, may comprise a second RFID tag 20B attached at the second attachment position proximate the second corner of the surgical sponge 10B with the longitudinal axis 21 of the RFID tag 20B oriented to be generally parallel to the second line L2. When the first surgical sponge 10A and second surgical sponge 10B are stacked on top of one another and the various longitudinal axes 21 of each RFID tag 20A, 20B are projected onto a common plane, the longitudinal axis 21 of the first RFID tag 20A and the second RFID tag 20B will be oriented in generally perpendicular configuration relative to one another when viewed from above. One of a plurality of advantages provided by this configuration is the reduced likelihood of interference when scanning the first and second surgical sponges 10A, 10B while stacked, increasing the read distance of the RFID tags 20, decreasing the likelihood of an error in counting and/or locating surgical sponges before, during, and after a surgical procedure.

Additionally, the RFID tag 20 of adjacent surgical sponges 10 may comprise similar attachment positions relative to the surgical sponge 10, but adjacent surgical sponge 10 may be arranged within the stack and/or packaging such that the RFID tags 20 of adjacent sponges 10 comprise different attachment positions relative to the packaging. For example, as illustrated in FIGS. 7A and 7B, a second surgical sponge 10B may comprise a second RFID tag 20B attached to the top surface 12 at the second attachment position of the surgical sponge 10B with the longitudinal axis 21 of the RFID tag 20B oriented to be generally parallel to the second line L2. A third surgical sponge 10C, placed adjacent the second surgical sponge 10B when packaged, may comprise a third RFID tag 20C attached to the top surface 12 at the third attachment position of the surgical sponge 10C with the longitudinal axis 21 of the RFID tag 20C oriented to be generally parallel to the first line L1. However, when the third surgical sponge 10C is stacked adjacent the second surgical sponge 10B, the third surgical sponge may be flipped 180 degrees about the first longitudinal axis L1. By flipping the third surgical sponge 10C, the third attachment position of third RFID tag 20C will be in an adjacent corner for the second attachment position of the second surgical sponge 20B relative to the packaging. When the second surgical sponge 10B and third surgical sponge 10C are stacked on top of one another and the various longitudinal axes 21 of each RFID tag 20B, 20C are projected onto a common plane, the longitudinal axis 21 of the second RFID tag 20B and the third RFID tag 20C will be oriented in generally perpendicular configuration relative to one another when viewed from above. Furthermore, the third RFID tag 20C of the third surgical sponge 10C will be in an adjacent corner (third attachment position) relative to the second RFID tag 20B of the first surgical sponge 10B (second attachment position).

When adding a fourth surgical sponge 10D to the stack, the orientation of the fourth RFID tag 20D may be different relative to the third RFID tag 20C of the third surgical sponge 10C. For example, as illustrated in FIGS. 7A and 7B, a third surgical sponge 10C may comprise a third RFID tag 20C attached to the top surface 12 at the third attachment position of the surgical sponge 10C with the longitudinal axis 21 of the RFID tag 20C oriented to be generally parallel to the first line L1. A fourth surgical sponge 10D, placed adjacent the third surgical sponge 10C when packaged, may comprise a fourth RFID tag 20D attached to the top surface 12 at a fourth attachment position of the surgical sponge 10D with the longitudinal axis 21 of the RFID tag 20D oriented to be generally parallel to the second line L2. The third and fourth surgical sponges 10C, 10D may be flipped approximately 180 degrees about the first line L1. By flipping the third and fourth surgical sponges 10C, 10D, the third RFID tag 20C and fourth RFID tag 20D, will have the appearance of being attached at an attachment position adjacent the first and second attachment positions of the first and second RFID tags 20A, 20B described above. Additionally, when the third surgical sponge 10C and fourth surgical sponge 10D are stacked on top of one another and the various longitudinal axes 21 of each RFID tag 20C, 20D are projected onto a common plane, the longitudinal axis 21 of the third RFID tag 20C and the fourth RFID tag 20D will be oriented in generally perpendicular configuration relative to one another when viewed from above.

As described above, the orientation and/or attachment position of the RFID tag 20 of a prior surgical sponge 10 included in bundle may be repeated in a subsequent surgical sponge 10, such that the orientation and/or attachment position of the adjacent RFID tag 20 should be different. For example, referring to FIGS. 7A and 7B, a fifth surgical sponge 10E may comprise a fifth RFID tag 20E attached at the fifth attachment position of the fifth surgical sponge 10E with the longitudinal axis 21 of the RFID tag 20E oriented to be generally parallel to the first line L1, similar to the first RFID tag 10A of the first surgical sponge 10A. However, the first surgical sponge 10A and the fifth surgical sponge 10E are not adjacent to one another when stacked and assembled in the packaging. The gap between the first and fifth surgical sponges 10A, 10E created by the intermediary surgical sponges 10B, 10C, 10D reduces the likelihood of interference between the first RFID tag 10A and fifth RFID tag 10E when scanned.

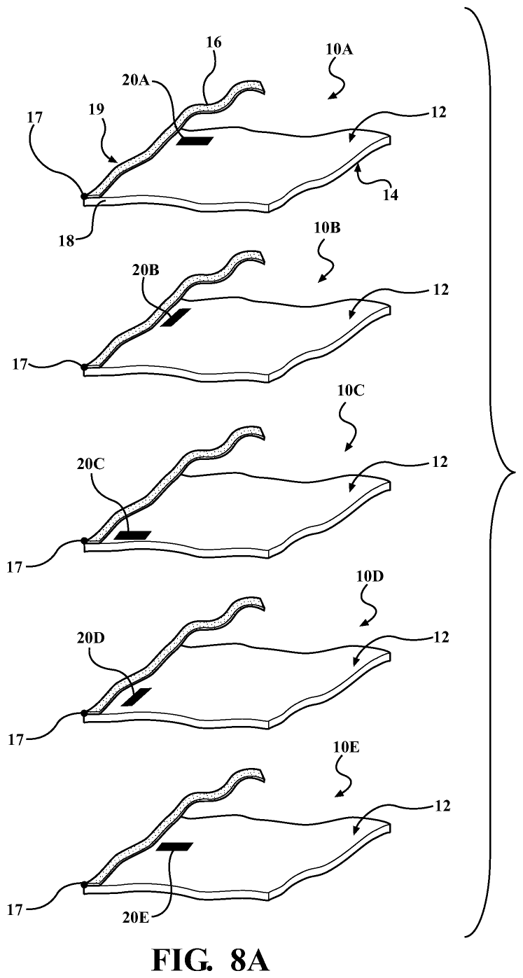

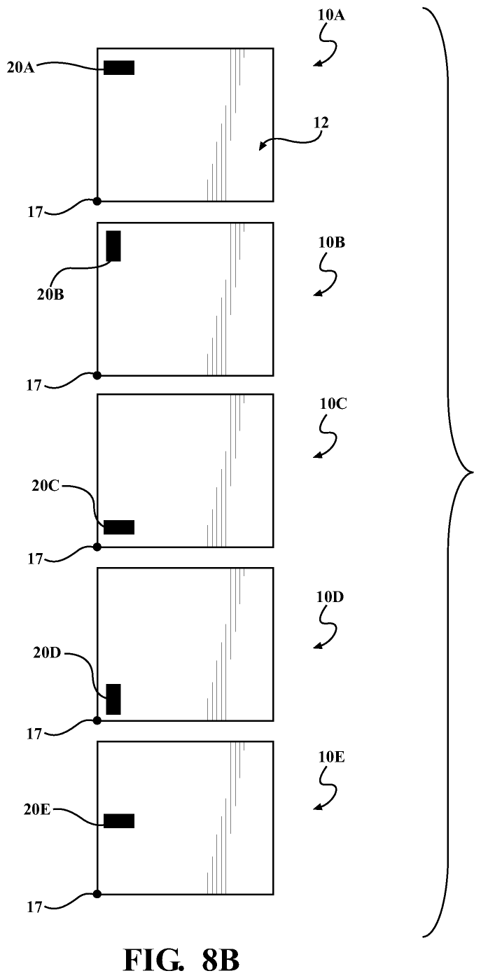

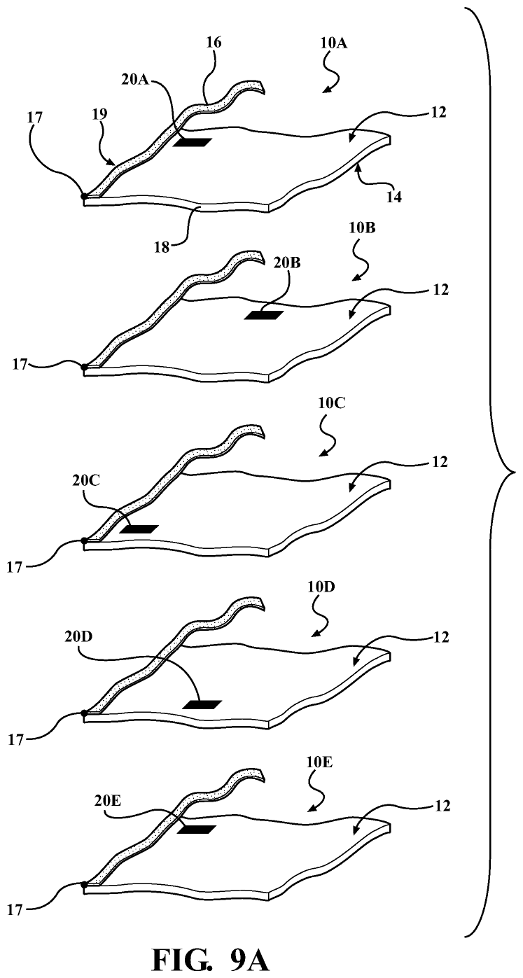

Referring to FIGS. 8A and 8B, an alternative embodiment of a configuration for packaging a plurality of surgical sponges 10 is illustrated. As described above, to prevent interference between the RFID tags 20 of adjacent surgical sponges 10 that have been stacked during packaging, the orientation of the RFID tag 20 may be different. For example, as illustrated in FIGS. 8A and 8B, a first surgical sponge 10A may comprise a first RFID tag 20A attached at the first attachment position of the surgical sponge 10A with the longitudinal axis 21 of the RFID tag 20A oriented to be generally parallel to the second line L2. A second surgical sponge 10B, placed adjacent the first surgical sponge 10A when packaged, may comprise a second RFID tag 20B attached at the second attachment position of the surgical sponge 10B with the longitudinal axis 21 of the RFID tag 20B oriented to be generally parallel to the first line L1. When the first surgical sponge 10A and second surgical sponge 10B are stacked on top of one another and the various longitudinal axes 21 of each RFID tag 20A, 20B are projected onto a common plane, the longitudinal axes 21 of the first RFID tag 20A and the second RFID tag 20B will be oriented in generally perpendicular configuration relative to one another when viewed from above. One of a plurality of advantages provided by this configuration is the reduced likelihood of interference when scanning the first and second surgical sponges 10A, 10B while stacked, decreasing the likelihood of an error in counting and/or locating surgical sponges before, during, and after a surgical procedure.

Additionally, the RFID tag 20 may be attached to adjacent or opposing corners of the surgical sponges 10 relative to the RFID tag 20 of an adjacent surgical sponge 10. For example, as illustrated in FIGS. 8A and 8B, a second surgical sponge 10B may comprise a second RFID tag 20B attached at the second attachment position of the surgical sponge 10B with the longitudinal axis 21 of the RFID tag 20B oriented to be generally parallel to the first line L1. A third surgical sponge 10C, placed adjacent the second surgical sponge 10B when packaged, may comprise a third RFID tag 20C attached at the third attachment position of the surgical sponge 10C with the longitudinal axis 21 of the RFID tag 20C oriented to be generally parallel to the second line L2. When the second surgical sponge 10B and third surgical sponge 10C are stacked on top of one another and the various longitudinal axes 21 of each RFID tag 20B, 20C are projected onto a common plane, the longitudinal axis 21 of the second RFID tag 20B and the third RFID tag 20C will be oriented in generally perpendicular configuration relative to one another when viewed from above. Furthermore, the third RFID tag 20C of the third surgical sponge 10C will be in an adjacent corner (third attachment position) relative to the second RFID tag 20B of the second surgical sponge 10B (second attachment position).

When adding a fourth surgical sponge 10D to the stack, the orientation of the fourth RFID tag 20D may be different relative to the third RFID tag 20C of the third surgical sponge 10C. For example, as illustrated in FIGS. 8A and 8B, a third surgical sponge 10C may comprise a third RFID tag 20C attached at the third attachment position of the surgical sponge 10C with the longitudinal axis 21 of the RFID tag 20C oriented to be generally parallel to the second line L2. The fourth surgical sponge 10D, placed adjacent the third surgical sponge 10C when packaged, may comprise a fourth RFID tag 20D attached at the fourth attachment position of the surgical sponge 10D with the longitudinal axis 21 of the RFID tag 20D oriented to be generally parallel to the first line L1. When the third surgical sponge 10C and fourth surgical sponge 10D are stacked on top of one another and the various longitudinal axes 21 of each RFID tag 20C, 20D are projected onto a common plane, the longitudinal axis 21 of the third RFID tag 20C and the fourth RFID tag 20D will be oriented in generally perpendicular configuration relative to one another when viewed from above.

The orientation of the RFID tag 20 and/or the attachment position of the RFID tag 20 on the surgical sponge 10 of adjacent surgical sponges 10 may be manipulated as described above to reduce interference when scanning for RFID tags 20 to count and or locate the corresponding surgical sponges 10. When adding additional surgical sponges 10 to the stack, the orientation and/or attachment position of the RFID tag 20 of a prior surgical sponge may be repeated in a subsequent surgical sponge 10, so long as, the orientation and/or attachment positions of the RFID tag 20 of adjacent sponges 10 should be different. For example, referring to FIGS. 8A and 8B, a fifth surgical sponge 10E may comprise a fifth RFID tag 20E attached at a fifth attachment position of the surgical sponge 10E, wherein the fifth RFID tag 20E is attached to the fifth surgical sponge 10E at an intermediate location between adjacent corners of the surgical sponge 10E with the longitudinal axis 21 of the RFID tag 20E oriented to be generally parallel to the second line L2. While the fifth RFID tag 20E is shown to be attached approximately half way between adjacent corners of the surgical sponge 10E, it is contemplated that the fifth RFID tag 20E may be placed at any point along the edge of the surgical sponge 10E between adjacent corners. It is also contemplated that the RFID tag 20 may be placed between opposing corners of the surgical sponge 10.

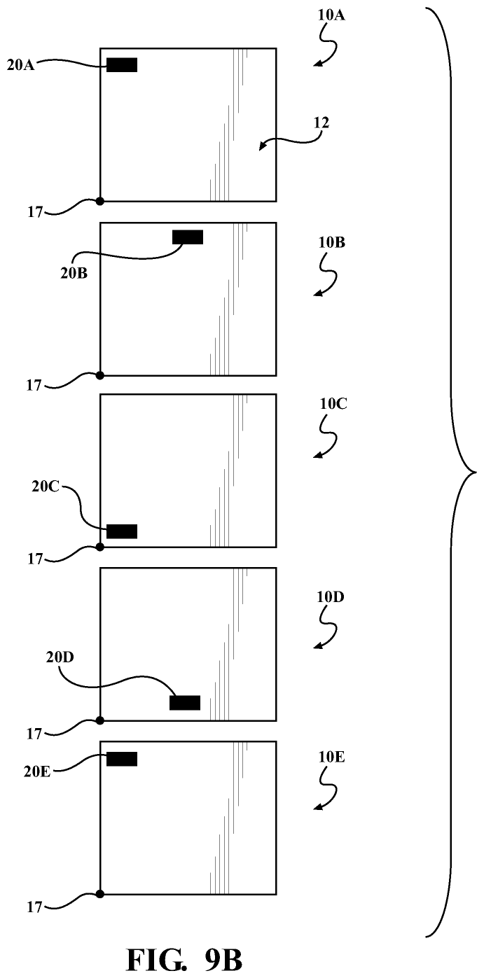

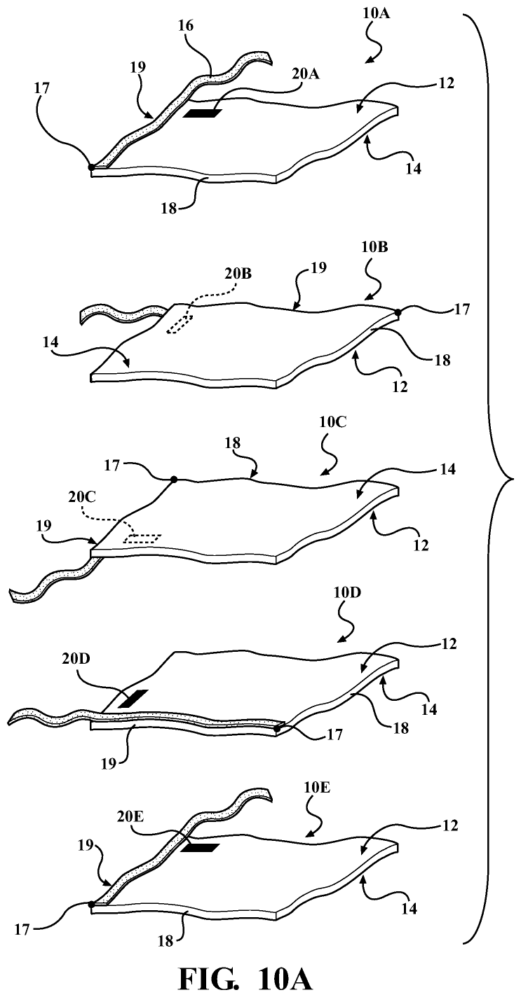

Referring to FIGS. 9A and 9B, an alternative embodiment of a configuration for packaging a plurality of surgical sponges 10 is illustrated. As described above, to prevent interference between the RFID tags 20 of adjacent surgical sponges 10 that have been stacked during packaging, the attachment position of the RFID tag 20 for one or more adjacent surgical sponges 10 may be different. For example, as illustrated in FIGS. 9A and 9B, a first surgical sponge 10A may comprise a first RFID tag 20A attached at the first attachment position of the surgical sponge 10A with the longitudinal axis 21 of the RFID tag 20A oriented to be generally parallel to the second line L2. A second surgical sponge 10B, placed adjacent the first surgical sponge 10A when packaged, may comprise a second RFID tag 20B attached at the second attachment position of the surgical sponge 10B with the longitudinal axis 21 of the RFID tag 20B oriented to be generally parallel to the second line L2. When the first surgical sponge 10A and second surgical sponge 10B are stacked on top of one another, the longitudinal axes 21 of each RFID tag 20A, 20B may be generally parallel when projected onto a common plane. While the longitudinal axis 21 of the first and second RFID tags may be substantially axially aligned, the attachment position of each RFID tag 20A, 20B may be different relative to one another when viewed from above. Staggering the attachment position of the RFID tags 20A, 20B, as illustrated in FIGS. 9A and 9B may reduce the likelihood of interference when scanning the first and second surgical sponges 10A, 10B while stacked, decreasing the likelihood of an error in counting and/or locating surgical sponges 10 before, during, and after a surgical procedure.

Additionally, the RFID tag 20 may be attached to adjacent or opposing corners of the surgical sponge 10 relative to the RFID tag 20 of one or more adjacent surgical sponges 10. For example, as illustrated in FIGS. 9A and 9B, a second surgical sponge 10B may comprise a second RFID tag 20B attached at the second attachment position of the surgical sponge 10B with the longitudinal axis 21 of the RFID tag 20B oriented to be generally parallel to the second line L2. A third surgical sponge 10C, placed adjacent the second surgical sponge 10B when packaged, may comprise a third RFID tag 20C attached at the third attachment position of the surgical sponge 10C with the longitudinal axis 21 of the RFID tag 20C oriented to be generally parallel to the second line L2. When the second surgical sponge 10B and third surgical sponge 10C are stacked on top of one another, the longitudinal axes 21 of each RFID tag 20B, 20C may be generally parallel to one another when projected onto a common plane. However, the second and third attachment positions of the second RFID tag 20B and the third RFID tag 20C respectively, may be positioned proximate to opposing sides of their respective sponges 10B and 10C when viewed from above. The distinct attachment positions of the second RFID tag 20B and the third RFID tag 20C may reduce the likelihood of interference when scanning the second and third surgical sponges 10B, 10C while stacked, decreasing the likelihood of an error in counting and/or locating surgical sponges 10 before, during, and after a surgical procedure.

When adding a fourth surgical sponge 10D to the stack, the attachment position of the fourth RFID tag 20D may be different relative to the third RFID tag 20C of the third surgical sponge 10C. For example, as illustrated in FIGS. 9A and 9B, a third surgical sponge 10C may comprise a third RFID tag 20C attached at the third attachment position of the surgical sponge 10C with the longitudinal axis 21 of the RFID tag 20C oriented to be generally parallel to the second line L2. The fourth surgical sponge 10D, placed adjacent the third surgical sponge 10C when packaged, may comprise a fourth RFID tag 20D attached at the fourth attachment position of the surgical sponge 10D with the longitudinal axis 21 of the RFID tag 20D oriented to be generally parallel to the second line L2. When the third surgical sponge 10C and fourth surgical sponge 10D are stacked on top of one another, the longitudinal axes 21 of each RFID tag 20C, 20D may be generally parallel when projected onto a common plane. While the longitudinal axis 21 of the third and fourth RFID tags may be substantially axially aligned, the attachment positions of the third RFID tag 20C and the fourth RFID tag 20D may be different from one another when viewed from above. Staggering the attachment positions of the third RFID tag 20C and the fourth RFID tag 20D may reduce the likelihood of interference when scanning the third and fourth surgical sponges 10C, 10D while stacked, decreasing the likelihood of an error in counting and/or locating surgical sponges before, during, and after a surgical procedure.

The attachment position of the RFID tag 20 on the surgical sponge 10 of adjacent surgical sponges 10 may be manipulated as described above to reduce interference when scanning for RFID tags 20 to count and/or locate the corresponding surgical sponges 10. When adding additional surgical sponges 10 to the stack, the attachment position of the RFID tag 20 of a prior surgical sponge may be repeated in a subsequent surgical sponge 10, so long as the attachment positions of the RFID tag 20 of the one or more adjacent sponges 10 should be different. For example, referring to FIGS. 9A and 9B, a fifth surgical sponge 10E may comprise a fifth RFID tag 20E that is attached at the fifth attachment position of the surgical sponge 10E with the longitudinal axis 21 of the RFID tag 20E oriented to be generally parallel to the second line L2, similar to the first surgical sponge 10A illustrated in FIGS. 9A and 9B. While the attachment position and orientation of the first surgical sponge 10A and fifth surgical sponge 10E of FIGS. 9A and 9B may be similar, the first surgical sponge 10A and the fifth surgical sponge 10E are not adjacent to one another when stacked and assembled in the packaging. The gap between the first and fifth surgical sponges 10A, 10E created by the intermediary surgical sponges 10B, 10C, 10D reduces the likelihood of interference between the first RFID tag 10A and fifth RFID tag 10E when scanned.

In addition to staggering the RFID tags 20 that may be substantially axially aligned, as described above, a different resonant frequency may be assigned and/or selected for the one or more adjacent surgical sponges 10 within a package. The combination of staggering axially aligned RFID tags 20 and selecting different resonant frequencies for one or more adjacent surgical sponges 10, reduces the likelihood of interference between one or more adjacent RFID tags 20 when scanned. FIG. 11 includes a graph illustrating an exemplary relationship between the distance between RFID tags 20 and the effective read distance for the scanner based on the resonant frequency of the RFID tags 20. The thick, solid line 52 represents the baseline of the read distance of a single tag by itself. The thin solid 54, dash-dot 56 and dash-double-dot 58 lines indicate the read distance of that tag when a 2^{nd} RFID tag, that has a higher resonant frequency, is placed coaxially behind it at different distances. The graph shows that there is an increase in the read distance which generally decreases as the 2^{nd} tag is moved away from the first tag. The dot 60 and dash 62 lines indicate the read distance of that tag when a 2^{nd} RFID tag, that has a lower resonant frequency, is placed coaxially behind it at different distances. The graph shows that there is a decrease in the read distance which generally increases as the 2^{nd} tag is moved away from the first tag. Based on this information we considered the possibility of sorting RFID tags and packaging them in pairs, each lower frequency tag with a higher frequency tag. These groups could be subdivided to further optimize the pairing for bundle read distance.

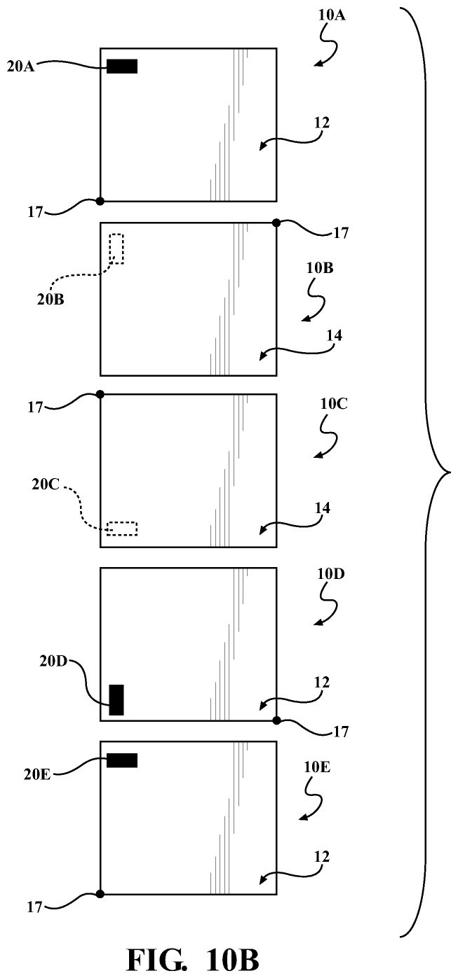

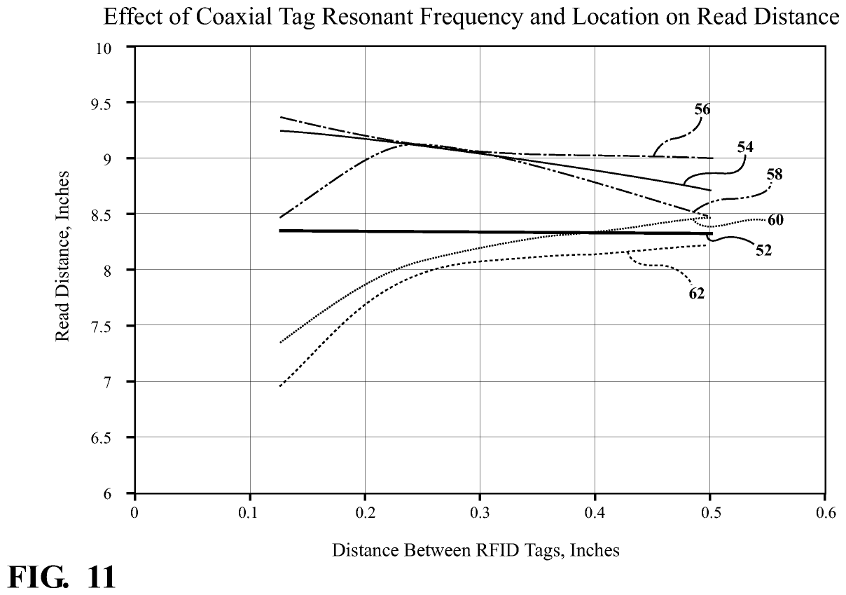

Referring to FIGS. 10A and 10B, an alternative embodiment of a configuration for packaging a plurality of surgical sponges 10 is illustrated. As described above, to prevent interference between the RFID tags 20 of adjacent surgical sponges 10 that have been stacked during packaging, the arrangement of the surgical sponge 10 within the packaging 38, 50 may be different. The surgical sponge 10 may be manipulated by flipping the surgical sponge 10 about an axis and/or rotating the surgical sponge 10 relative to one or more adjacent surgical sponges 10. Said manipulation of the surgical sponge 10 prior to packaging can provide the appearance of a different orientation and/or attachment position for the RFID tag 20 of one or more adjacent surgical sponges 10 relative to the packaging 38, 50. For example, as illustrated in FIGS. 10A and 10B, a first surgical sponge 10A may comprise a first RFID tag 20A attached at the first attachment position of the surgical sponge 10A with the longitudinal axis 21 of the RFID tag 20A oriented to be generally parallel to the second line L2. A second surgical sponge 10B, placed adjacent the first surgical sponge 10A when packaged, may comprise a second RFID tag 20B attached at the second attachment position of the surgical sponge 10B with the longitudinal axis 21 of the RFID tag 20B oriented to be generally parallel to the second line L2. The first and second attachment positions and/or orientations of the RFID tags 20A and 20B relative to the surgical sponge 10A, 10B may be the same or substantially similar. However, the second surgical sponge 10B, starting from the same position as the first surgical sponge 10A, may be flipped about an axis, such as the axis created by the first line L1, and rotated approximately 90 degrees about the origin point 17. By flipping and rotating the second surgical sponge 10B, the longitudinal axis 21 of the first RFID tag 10A (generally parallel to the second line L2) and the second RFID tag 10B (generally parallel to the first line L1) are now different when projected onto a common plane. This configuration may reduce the likelihood of interference when scanning the first and second surgical sponges 10A, 10B while stacked, decreasing the likelihood of an error in counting and/or locating surgical sponges before, during, and after a surgical procedure.

The surgical sponge 10 may be further manipulated relative to the packaging 38, 50 to provide additional orientation and/or attachment positions of one or more adjacent surgical sponges 10. For example, as illustrated in FIGS. 10A and 10B, a second surgical sponge 10B has been flipped and rotated to provide configuration wherein the second RFID tag 20B is attached at the second attachment position of the surgical sponge 10B with the longitudinal axis 21 of the RFID tag 20B oriented to be generally parallel to the first line L1. A third surgical sponge 10C, placed adjacent the second surgical sponge 10B when packaged, may also be flipped and/or rotated relative to the packaging 38, 50 to create the appearance of an attachment position and/or orientation of the RFID tag 20C that is distinct from one or more adjacent surgical sponges 10. As illustrated in FIGS. 10A and 10B, the third surgical sponge 10C, starting from the same position as the first surgical sponge 10A, may be flipped about an axis, such as the axis created by the first line L1, and rotated approximately 180 degrees. By flipping and rotating the third surgical sponge 10C, the longitudinal axis 21 of the second RFID tag 10B (generally parallel to the first line L1) and the third RFID tag 10C (generally parallel to the second line L2) are now different when projected onto a common plane. Furthermore, the attachment position of the RFID tags 20B, 20C are now in adjacent corners when the sponges are stacked and view from above, relative to the packaging 38, 50. This configuration may reduce the likelihood of interference when scanning the second and third surgical sponges 10B, 10C while stacked, decreasing the likelihood of an error in counting and/or locating surgical sponges 10 before, during, and after a surgical procedure.

When adding a fourth surgical sponge 10D to the stack, may be further manipulated relative to the packaging 38, 50 to provide additional orientation and/or attachment positions of one or more adjacent surgical sponges 10. For example, as illustrated in FIGS. 10A and 10B, a third surgical sponge 10C may be flipped and/or rotated to comprise a third RFID tag 20C attached at the third attachment position of the surgical sponge 10C with the longitudinal axis 21 of the RFID tag 20C oriented to be generally parallel to the second line L2. The fourth surgical sponge 10D, starting from the same position as the first surgical sponge 10A, may be rotated approximately 90 degrees about the origin point 17. By rotating the fourth surgical sponge 10D, the longitudinal axis 21 of the third RFID tag 10C (generally parallel to the second line L2) and the fourth RFID tag 10D (generally parallel to the first line L1) are now different when projected onto a common plane. This configuration may reduce the likelihood of interference when scanning the third and fourth surgical sponges 10C, 10D while stacked, decreasing the likelihood of an error in counting and/or locating surgical sponges before, during, and after a surgical procedure.

The surgical sponge 10 may be manipulated relative to one or more adjacent surgical sponges 10 as described above to reduce interference when scanning for RFID tags 20 to count and or locate the corresponding surgical sponges 10. When adding additional surgical sponges 10 to the stack, the surgical sponge 10 may be manipulated relative to the packaging 38, 50 to provide a distinct orientation and/or attachment position of the RFID tag 20 when projected onto a common plane. The process of manipulating a surgical sponge 10 by flipping and/or rotating a prior surgical sponge 10 may be repeated for a subsequent surgical sponge 10, so long as, the resulting orientation and/or attachment positions of the RFID tag 20 relative to the packaging 38, 50 for one or more adjacent sponges 10 should be different. While the attachment position and orientation of the RFID tags 20A, 20E of the first surgical sponge 10A and fifth surgical sponge 10E of FIGS. 10A and 10B may be similar, the first surgical sponge 10A and the fifth surgical sponge 10E are not adjacent to one another when stacked and assembled in the packaging 38, 50. The gap between the first and fifth surgical sponges 10A, 10E created by the intermediary surgical sponges 10B, 10C, 10D reduces the likelihood of interference between the first RFID tag 10A and fifth RFID tag 10E when scanned. While the manipulation of the surgical sponge 10 has been described in term so flipping the surgical sponge about the axis created by the first line L1, it should be understood that it is contemplated that the surgical sponge may be flipped about any axis. Furthermore, it is contemplated that the surgical sponge 10 may be rotated any number of degrees about the origin point 17 to provide a distinct RFID tag 20 attachment position and/or orientation. For example, the sponge may be rotated 45 degrees, 90 degrees, or 180 degrees in any direction about the origin point 17.

While the embodiments described above include a surgical sponge 10 with an RFID tag 20, the use of other counting and/or detecting elements is contemplated. In another embodiment, as an alternative to an RFID tag 20, the counting and/or detecting element may comprise an EAS element that cooperates with the detecting antenna of the reader to detect the location of the surgical article. In contrast to the RFID element, certain embodiments of the EAS element may not be used to uniquely identify the surgical article because these elements may be free from the unique identification information for the surgical article. However, similar to the RFID element, the EAS element may be encapsulated to provide biocompatible contact surfaces, water-resistance and/or sterilizability. For example, the EAS element can be enclosed in glass, polymer or a silicone pot, which can permit the repeated sterilization by various suitable methods using heat, gas, chemicals, or gamma radiation. Furthermore, the EAS elements may be incorporated into the tags, the handle, or other portions of the surgical article in any number of ways. For example, the EAS element can be adhered to or encapsulated within the tags, embedded within the handle, or coupled to other portions of the article. Advantageously, the EAS elements can be made quite as compact, for example, about 11 mm by 2.5 mm. Moreover, the compact construction of the EAS elements can be attached to correspondingly small articles such as hemostats, scalpel handles, and possibly the 4″ by 4″ gauze pads. Additionally, the EAS elements can be relatively inexpensive to manufacture and be less shielded by intervening tissue than the RFID elements. In one embodiment, the article can have diametrically opposite sides or corners, and two EAS elements may be coupled to those opposing corners of the article. The EAS element can generally operate in 500 kHz, but more preferably in the range of from 30 kHz to the range from 100 kHz to 150 kHz. Some embodiments of EAS elements can include: an acousto-magnetic element; an electro-magnetic element; a swept RF element; a LF (LC tank circuit) element; or any combination thereof. The EAS element may utilize a frequency range of from 10 Hz to 10 MHz. The EAS element of a missing or unaccounted-for article can be detected within the patient's body and other locations within the operating room.

The method of packaging a plurality of sponges may comprise attaching RFID tags 20 to surgical sponges 10 in one of the various example embodiments described above, and illustrated in FIGS. 5A-10B. For example, attaching a first RFID tag 20 to a first surgical sponge 10A at a first attachment position proximate a first corner of the surgical sponge 10 with the elongated side 22 oriented along a first longitudinal axis L1. Then attaching a second RFID tag 20B to a second surgical sponge 10B, that may be placed adjacent the first surgical sponge 10A when packaged, at the first attachment position proximate the first corner of the surgical sponge 10B with the elongated side 22 oriented along a second longitudinal axis L2. The first and second surgical sponges 10A, 10B may be assembled into a first stack, such that when the first surgical sponge 10A and second surgical sponge 10B are stacked on top of one another and the various longitudinal axes of each RFID tag 20 are projected onto a common plane, the first RFID tag 20A and the second RFID tag 20B will be oriented in generally perpendicular configuration relative to one another when viewed from above. The stack may then be secured by a strap, ribbon, or similar packaging configured to define a space within for securing the surgical sponges 10 in the provided configuration. The pattern of alternating the orientation of adjacent sponges may be continued, and additional sponges 10 may be stacked and/or secured together. For example, 5, 10, 20 . . . n-number of sponges 10 may be stacked and/or bundled together.

Alternatively, in addition to rotating the orientation of the RFID tag 20 between various longitudinal axes, the RFID tag 20 of adjacent surgical sponges 10 may be attached at different attachment positions. Different attachment positions may comprise attaching RFID tags 20 at adjacent corners of the surgical sponge 10. It may also include attaching RFID tags 20 at an intermediary attachment position located between two adjacent corners of the surgical sponge 10. For example, the method may comprise attaching the second RFID tag 20B at the first attachment position proximate a first corner of the surgical sponge 10 and attaching a third RFID tag 20C at a second attachment position proximate a second corner of the surgical sponge 10. Therefore, when the second surgical sponge 10B and third surgical sponge 10C are stacked on top of one another and the various longitudinal axes of each RFID tag 20 are projected onto a common plane, the second RFID tag 20B and the third RFID tag 20C will be oriented in generally perpendicular configuration relative to one another when viewed from above. Furthermore, the third RFID tag 20C of the third surgical sponge 10C will be in an adjacent corner (second corner) relative to the first RFID tag 20B of the second surgical sponge 10B. The stacked sponges 30 may then be similarly bundled, packaged, and/or secured as described above.

The method may further comprise manipulating the arrangement of one or more surgical sponges 10 within the stack 30 to create the appearance of the RFID tag 20 being attached at a different attachment position and/or orientation with respect the RFID tag of one or more adjacent surgical sponges 10. For example, the method may comprise attaching a second RFID tag 20B to the top surface 12 of the surgical sponge 10B at the second attachment position proximate the second corner of the surgical sponge 10B with the elongated side 22 oriented along the second longitudinal axis L2. A third RFID tag 20C may be attached to the top surface 12 of the surgical sponge 10C at the second attachment position proximate the second corner of the surgical sponge 10C with the elongated side 22 oriented along the first longitudinal axis L1. The second and third surgical sponges 10B, 10C may be assembled into a stack, wherein the step of assembling comprises flipping or rotating the third surgical sponge 10C approximately 180 degrees about the first longitudinal axis L1. By flipping the third surgical sponge 10C, the third RFID tag 20C, while attached at the second attachment position, it will have the appearance of being attached at a third attachment position proximate a third corner of the surgical sponge 10 when packaged. When the second surgical sponge 10B and third surgical sponge 10C are stacked on top of one another and the various longitudinal axes of each RFID tag 20 are projected onto a common plane, the second RFID tag 20B and the third RFID tag 20C will be oriented in generally perpendicular configuration relative to one another when viewed from above. Furthermore, the third RFID tag 20C of the third surgical sponge 10C will be in an adjacent corner (second corner) relative to the first RFID tag 20A of the first surgical sponge 10A. The stacked sponges 30 may then be similarly bundled, packaged, and/or secured as described above.

In order to perform one or more of the functions as described above, each tag 20 may comprise one or more counting elements, such as the optically-scannable element and the human-readable element, and one or more detecting elements, such as the RFID tag and the EAS element, or combinations thereof. It is possible that multiple tags, handles, or other portions of the surgical article are selected to have complementary features, such that, in combination on a given article, the two tags have counting elements and detecting elements that can perform all the functions described above. Further still, it is possible that certain functions are redundant across multiple tags as also described above. For example, it is possible that two tags are capable of communicating the same unique identification information for the corresponding surgical article. It is also contemplated that the surgical article may include tags, handles, or other portions of the surgical article having counting/detecting elements that are only capable of collectively performing only one, two, three or four of the functions outlined above.

The above disclosure encompasses multiple embodiments of packs of RFID-tagged surgical sponges and methods of packaging RFID-tagged sponges, to reduce or eliminate the risk of interference with reading or acquiring an RFID signal from each individual tagged sponge. In addition to configuring the sponge, properly configuring the scanning device may also work to reduce the risk of interference by ensuring high signal quality. The scanning device includes a radio signal generator coupled to an antenna through a tuning circuit. In some embodiments, the antenna may be a loop antenna. In order for a loop antenna to function correctly, the antenna needs to be both electrically resonant and have an impedance matched to the signal source, that is, the signal generator. The desired electrical resonance and impedance matching may be achieved by tailoring the characteristics of the tuning circuit. In accordance with the present disclosure, capacitors of various values and configurations are introduced into the electrical pathway connection the signal source and the antenna. It should be appreciated that the concepts described here may be used for other medical objects, such as other disposable medical items, including but not limited to cutting tools, sharps, wearable items, etc.

Conventional tuning methods are time and work intensive processes that involve interpreting the output of a device while manually placing and removing shunting jumpers onto vertical header connectors on a circuit board. Shunting jumpers are used to electrically connect or exclude components to vary the network performance characteristics being measured, for example, by a network analyzer. The network analyzer can provide information about the complex impedance of the circuit, or as a measure of the standing wave ratio. In one example embodiment, a 13.56 MHz RFID signal is preferably coupled to a loop antenna having an effective impedance of 50 Ohms. By providing an antenna tuning circuit having an appropriate tuning between the signal generator and the antenna loop, variability in the material and manufacturing of the antenna, and in the environment where the device is operated, can be accommodated without noticeable loss in the communication quality. For every device, a tuning process is performed where the component configuration is varied until the desired characteristics, as measured by the network analyzer, are achieved.

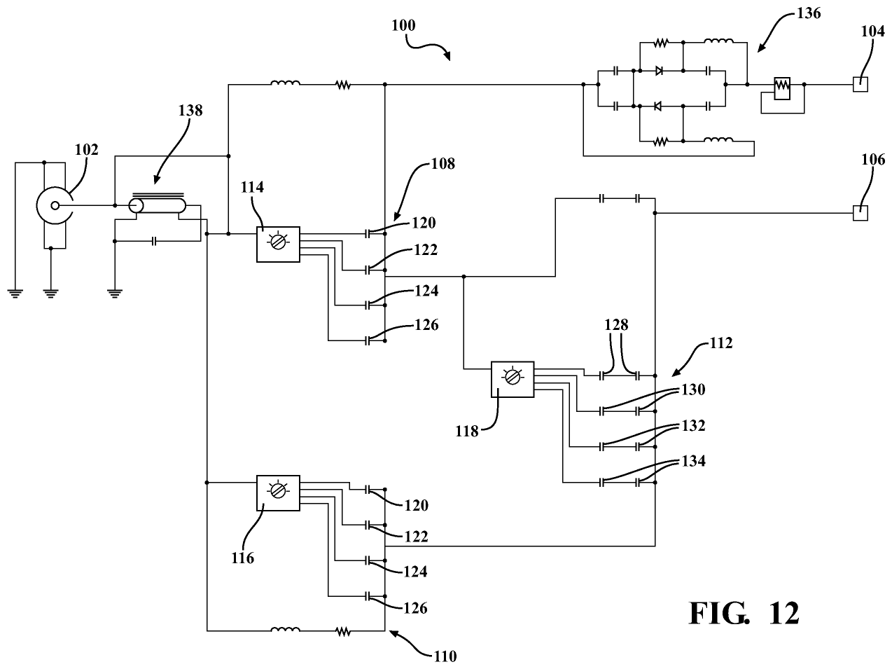

The present disclosure provides an improved circuit configuration that addresses the challenges of achieving the desired electrical resonance and impedance matching. As shown in FIG. 12, an exemplary tuning circuit 100 is provided. The tuning circuit includes a connector 102 for connection to the signal generator. In the illustrated embodiment, a co-axial connector style is shown, but any suitable connector may be used. The tuning circuit also includes connectors 104, 106 for connection to the antenna loop. In the illustrated embodiment, threaded, self-clinching studs are provided to both mechanically secure the antenna loop and electrically connect the antenna to the tuning circuit.

Between the first connector 102 for connection to the signal generator and the opposite connectors 104, 106 for connection to the antenna, capacitor banks 108, 110, 112 are disposed for conditioning the electrical characteristics of the circuit connecting the antenna to the signal generator. Each of the capacitor banks 108, 110, 112 includes capacitors connected to a rotary, binary-coded switch 114, 116, 118.

In the illustrated embodiment, each of the capacitor banks 108, 110 include four capacitors 114, 116, 118, 120, where each capacitor 114, 116, 118, 120 has a different capacitance value from the others in the bank. For example, capacitor 114 has a capacitance of 3.9 picofarads; capacitor 116 has a capacitance of 2 picofarads; capacitor 118 has a capacitance of 1 picofarad; and capacitor 120 has a capacitance of 0.5 picofarads. However, the number of capacitors, and the particular capacitance is not particularly limited.

The capacitor banks 108, 110 are primarily involved in modulating the effective impedance of the antenna on the signal generator. By selectively including and excluding certain capacitors from the tuning circuit the effective impedance can be changed. The rotary, binary coded switches 114, 116, having a 1-×-4 pin configuration, allows each combination of one or more of the capacitors to be sequentially included in the circuit for analyzing its performance, for example, with a network analyzer. The rotary binary coded switches eliminate the need for the manually intensive process of installing and removing shunting jumpers during the tuning process.

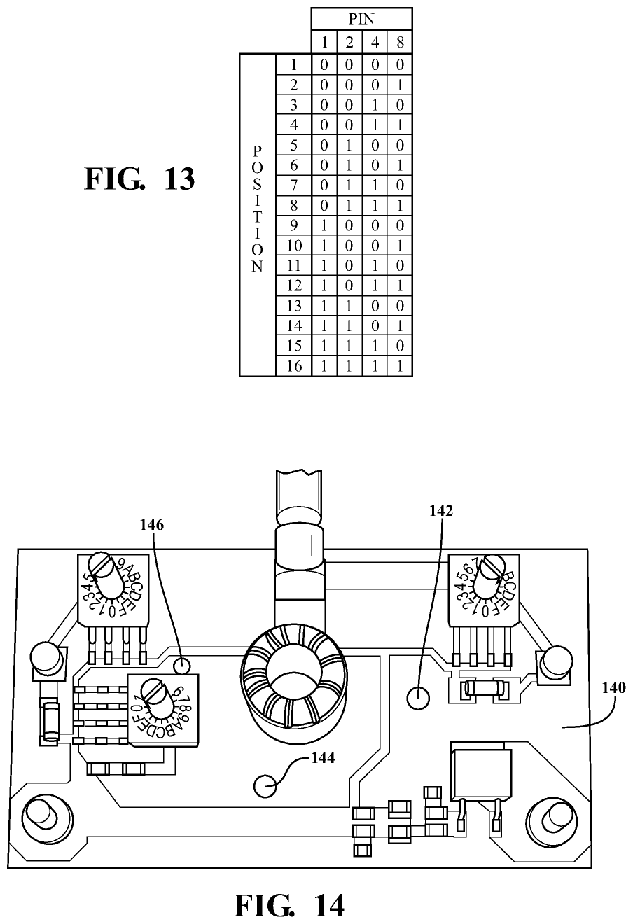

The rotary binary coded switch in the illustrated embodiments includes a 1-×-4 pin configuration and has 16 different switch positions, including one position where none of the capacitors are electrically active in the circuit and one position where all four of the capacitors are electrically active in the circuit. The remaining positions of the switch proceed in a binary coded fashion through the various permutations of capacitor connections. This sequence is illustrated in the table shown in FIG. 13. The table indicates the four pins, 1, 2, 4, 8, connected to the four capacitors, respectively, against the sixteen possible positions of the rotary switch. In the illustrated table a “0” indicates that the circuit is “open” for that pin, while a “1” indicates that the circuit is closed. In this way, during the antenna tuning process, the impact on circuit characteristics can be quickly and efficiently evaluated when operated in connection with a network analyzer. Other pin configurations and switch positions also possible for the rotary binary coded switch are further contemplated.

The capacitor bank 112 is primarily involved in modulating the antenna resonance. Like the capacitor banks 108 and 110, the bank 112 includes a plurality of capacitors arranged for selected inclusion in the tuning circuit 100 by way of a rotary binary coded switch 118 having a 1-×-4 pin configuration. On each of the four pin branches extending in parallel from the switch 118, two capacitors are arranged in series with each other. The capacitors on each branch have different capacitances. In one example embodiment, the capacitance of capacitor 128 is 7.5 picofarads; capacitor 130 is 3.9 picofarads; capacitor 132 is 2 picofarad; and capacitor 134 is 1 picofarad. As the rotary switch 118 is cycled through its multiple positions, connections are made according to the pattern shown in FIG. 13.

The tuning circuit 100 may include other operational circuit sections that provide additional functionality or otherwise achieved desired performance characteristics. For example, a high-frequency switch 136 may be included in the tuning circuit 100. The high-frequency switch may include a pair of anti-parallel PIN-diodes arranged so that, upon applying a DC-bias to the tuning circuit, the connection between the RF signal generator and the antenna loop will act as on open circuit. This allows rapid switching between actively transmitting a signal, and cutting off the signal transmission. The tuning circuit may further include a transmission line transformer 138, such as a balun, to transform an unbalanced RF signal into a balanced signal.

FIG. 14 illustrates an exemplary circuit board assembly 140 including the circuit shown in FIG. 12. The circuit board assembly 140 is a multi-layer printed circuit board including surface mounted components, and includes mechanical alignment holes 142, 144, 146. Although circuit board assembly 140 is shown with three alignment holes 142, 144, 146, more or fewer alignments holes may be used and/or may be located at different positions than shown. In further alternative embodiments, alignments features other than holes maybe employed, including for example, studs or optically recognizable fiducials or patterns.

The alignment features facilitate the reliable and repeatable interface between a circuit board assembly 140 and a tuning fixture (not shown). Where prior tuning processes required the manually intensive steps of repeatedly installing and removing shunting jumpers, the improved circuit according to the present disclosure avoids this requirement through the use of the rotary binary coded switches, rotatable by a tuning fixture.

The tuning fixture is a production machine that can mechanically connect to the rotary switches 114, 116, 118, and electrically connect to the tuning circuit, to automatically perform the tuning process. A signal generator and a network analyzer are included in the tuning fixture to generate a radio frequency signal and to monitor the circuit characteristics. The tuning fixture also includes mechanical manipulators which interface with the rotary switches and include motors to rotate the switches through their multiple positions. The tuning fixture further includes a controller in communication with the signal generator, network analyzer, and the mechanical manipulators for controlling an automated tuning process. The controller may further include a data logger to monitor and maintain a record of the execution of the tuning process and any measurements recorded during the execution.

The automatic tuning process performed by the tuning fixture may be configured to operate in different alternative embodiments. In a first embodiment, the automated tuning process is configured to evaluate and measure the standing wave ratio (SWR) at every permutation of the switch positions while a radio signal is applied to the tuning circuit. The tuning fixture records which switch positions achieve the lowest SWR value for the circuit and sets the switches at the corresponding positions for the lowest SWR value at the end of the tuning process.

In an alternative embodiment, a threshold SWR value may be predetermined for a given antenna configuration and anticipated operational environment. The predetermined SWR value may be empirically determined by testing the product configuration in an environment in accordance with desired performance characteristics. For example, it may be desirable to ensure that the effective read range for counting-in RFID tagged sponges is at least 12 inches away from the scanning device, wherein the scanning device includes a loop antenna having a diameter of 12 inches. Achieving consistent RF communication between the scanning device and the RFID tagged sponge may require a maximum SWR threshold value. The automatic tuning process may thus use the determined threshold SWR value and cycle the rotary switches through different positions until the first value lower than the threshold SWR value is reached, after which point the tuning process is terminated. The resulting SWR value and corresponding switch positions may be recorded into a memory of the tuning fixture.

In a further alternative embodiment, the tuning fixture may be configured to evaluate the total complex impedance, the SWR value, or a combination of the two values, in order to determine whether more or less capacitance was required in one or more of the capacitor banks. Based on that determination, the manipulators would thus adjust the particular rotary switch to a particular position corresponding in order to known capacitor values to achieve the desired network performance. Although described separately, a further alternative of the tuning fixture may be configured to operate as a hybrid or combination of this embodiment and one or more of the prior described alternatives.

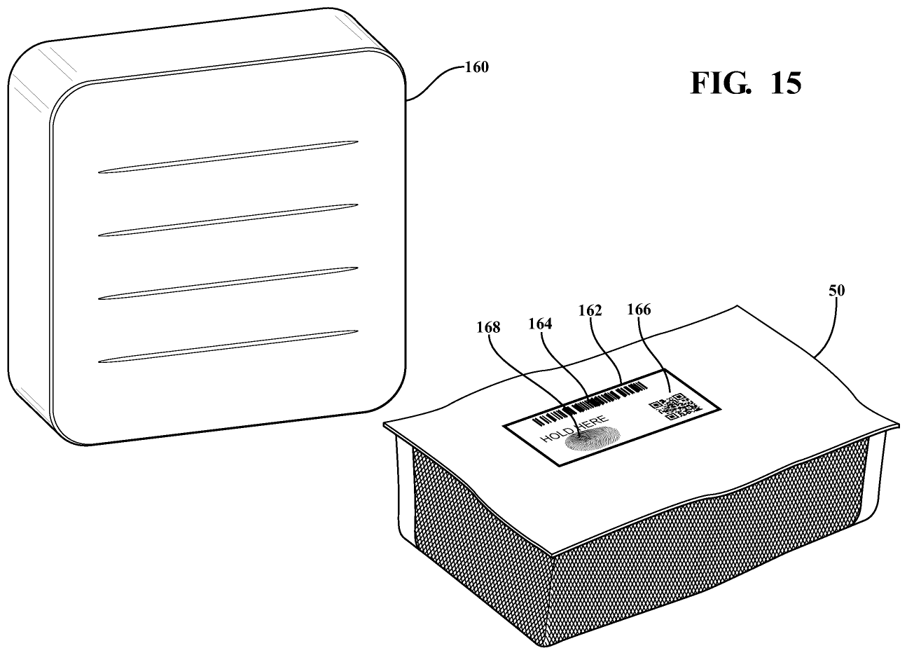

In the desire to ensure consistent, high quality RF communication between a scanning device and an RFID tag, whether on the sponge or on packaging, it may be desirable to provide user instruction directly on the tagged object. Positioning a human body, or parts thereof, between a transmitter's antenna and a receiver's antenna can cause degradation in communication performance. As illustrated in FIG. 15, container 50, as an example of a tagged object, is shown positioned relative to a scanning device 160. The tagged object includes an RFID tag 162, which may be a master tag as described above. Where the tagged object is a tagged surgical article, such as a sponge, the tag 162 may be an article tag such as tag 20. The tag 162 may be mounted on the object and may further include human or machine, optically-readable information beyond the RFID components embedded or encapsulated within. The information may for example be display as a bar code 164, or a quick response (QR) code 166, i.e. a two-dimensional bar code. The tag 162 may also include human readable text (not shown) that identifies the object, the manufacturer or distributor of the object, a unique identifier, combinations thereof, and the like.

Beyond the product information printed on the tag, instruction information 168 for aiding in the scanning process may also be positioned on the tagged object, such as being printed on the tag, or other location on the tagged object, which may be the surgical sponge or a package containing surgical sponges. For example, this instruction information 168 may be provided to designate where an operator should hold, pinch, or touch the object during scanning, i.e., a holding area. This instruction information may include a shape, such as a circle or oval, a pattern, such as a target reticle or fingerprint to indicate where the user should grasp the tagged object. The indication may further include instruction text, such as ‘hold here,’ ‘pinch here,’ ‘touch here,’ combinations of one or more such indications, and the like. In addition to the printed shape and/or instructions, the indication may be a textured area on the object that the user may quickly identify without looking at the object.

In a further example, the instruction information 168 may include the text ‘pinch here’ printed on the tag 162, with a raised or textured fingerprint pattern on the tag 162 adjacent the text, and further include a second raised or textured fingerprint pattern on the opposite surface of the object, e.g., the package 50. The operator, during the scanning operation, may see and/or feel the textured pattern of the instruction 168 on the tag 162 and, while holding the object, locate the textured pattern on the opposite surface of the object, in this case—package 50, and thus hold the package in the preferred orientation to minimize any interference between the tag 162 and the scanning device 160.

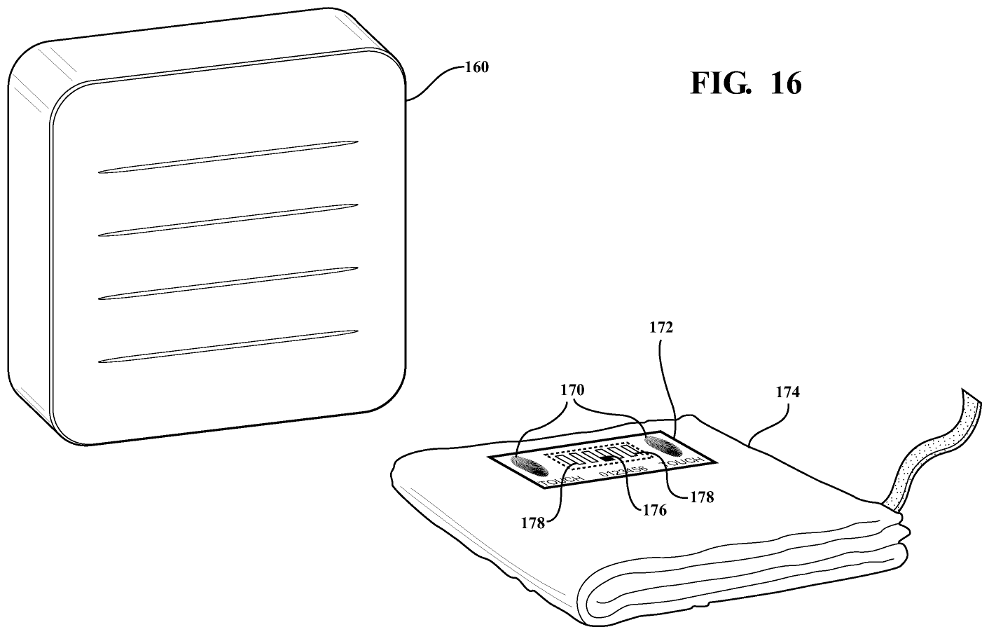

In another example, shown in FIG. 16, the instruction information 170 may include the text ‘touch here’ printed on a tag 172 coupled to the object, in this example sponge 174. The instruction information 170 includes two holding area locations that the user may bridge across with their fingers, or may touch one or the other of the holding areas. The tag 172 further includes an RFID chip 176 and antenna 178 encapsulated within the tag 172. In the illustrated embodiment, the tag includes a dipole antenna that may benefit from the presence of the user's hand at one or both ends to counteract any deleterious impact of saline or other environmental factors on the read range.

As will be understood by those of skill in the art, the beneficial configuration of holding area to antenna or tag design may depend on the particular type of tag chosen, e.g. UHF or HF RFID, on the environment in which the tag is employed, on the scanning device design, and other routine engineering considerations. The beneficial configuration can be determined empirically for a particular product system by testing the effective read range in various configurations of holding areas. For example, in an UHF RFID system, a beneficial configuration may include providing holding areas so that the antenna extends in parallel to scanning device. In an HF RFID, a beneficial configuration may include the antenna extending perpendicular to the scanning device.

Positioning instruction information with an indication how to hold during scanning on the tagged object helps ensure that the tagged object is optimally oriented relative to the scanner, ensures that any negative impact from human body radio interference is minimized, and can help to ensure that the RF communication between the tagged object and the scanning device occurs without reducing read range.

A method of using an RFID sponge may include retrieving the tagged object from its packaging. Prior to using the tagged object in the surgical field, the operator would locate the information provided on the object, including an indication for the prior orientation and holding position during a count-in scan. Holding the object in the holding area, the operator would scan in the object to enable its identification and tracking during the surgical operation. Once scanned in, the tagged object would then be used in the surgical operation. Likewise, during a count-out scan at the conclusion of the surgical operation, the indication would inform the operator of the preferred orientation and holding area to ensure proper communication with the scanning device.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. An RFID sponge, comprising:
an absorbent material;
a tag coupled to the absorbent material, the tag comprising an RFID element; and
instruction information disposed on the absorbent material designating a holding area.
